(19) United States
(12) Patent Application Publication
(10) Pub. No.: US 2009/0170882 A1
(43) Pub. Date: Jul. 2, 2009

(54) Methods and compositions

(76) Inventors: Dashyant Dhanak, Collegeville, PA (US); Steven David Knight, Collegeville, PA (US); Michael Lee Moore, Collegeville, PA (US); Kenneth Alan Newlander, Collegeville, PA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/631,138

(22) PCT Filed: Jun. 30, 2005
(86) PCT No.: PCT/US05/23758

§ 371 Date: Nov. 13, 2008

Related U.S. Application Data
(60) Provisional application No. 60/584,842, filed on Jun. 30, 2004.

(51) Int. Cl. A61K 31/403; C07D 209/88; A61K 31/519; A61P 37/00; A61P 29/00; A61P 31/10; A61P 35/00; C07D 487/04

(52) U.S. Cl. 514/267; 548/440; 544/250; 514/411

(57) ABSTRACT

Compounds, compositions and methods relating to kinesin inhibition are described herein.

### FIELD OF THE INVENTION

This invention relates to compounds which are inhibitors of the mitotic kinesin KSP and are useful in the treatment of cellular proliferative diseases, for example cancer, hyperplasias, restenosis, cardiac hypertrophy, immune disorders, fungal disorders, and inflammation.

### BACKGROUND OF THE INVENTION

Among the therapeutic agents used to treat cancer are the taxanes and vinca alkaloids, which act on microtubules. Microtubules are the primary structural element of the mitotic spindle. The mitotic spindle is responsible for distribution of replicate copies of the genome to each of the two daughter cells that result from cell division. It is presumed that disruption of the mitotic spindle by these drugs results in inhibition of cancer cell division, and induction of cancer cell death. However, microtubules form other types of cellular structures, including tracks for intracellular transport in nerve processes. Because these agents do not specifically target mitotic spindles, they have side effects that limit their usefulness.

Improvements in the specificity of agents used to treat cancer is of considerable interest because of the therapeutic benefits which would be realized if the side effects associated with the administration of these agents could be reduced. Traditionally, dramatic improvements in the treatment of cancer are associated with identification of therapeutic agents acting through novel mechanisms. Examples of this include not only the taxanes, but also the camptothecin class of topoisomerase I inhibitors. From both of these perspectives, mitotic kinesins are attractive targets for new anti-cancer agents.

Mitotic kinesins are enzymes essential for assembly and function of the mitotic spindle, but are not generally part of other microtubule structures, such as in nerve processes. Mitotic kinesins play essential roles during all phases of mitosis. These enzymes are “molecular motors” that transform energy released by hydrolysis of ATP into mechanical force which drives the directional movement of cellular cargoes along microtubules. The catalytic domain sufficient for this task is a compact structure of approximately 340 amino acids. During mitosis, kinesins organize microtubules into the bipolar structure that is the mitotic spindle. Kinesins mediate movement of chromosomes along spindle microtubules, as well as structural changes in the mitotic spindle associated with specific phases of mitosis. Experimental perturbation of mitotic kinesin function causes malformation or dysfunction of the mitotic spindle, frequently resulting in cell cycle arrest and cell death.

Among the mitotic kinesins which have been identified is KSP. KSP belongs to an evolutionarily conserved kinesin subfamily of plus end-directed microtubule motors that assemble into bipolar homotetramers consisting of antiparallel homodimers. During mitosis KSP associates with microtubules of the mitotic spindle. Microinjection of antibodies directed against KSP into human cells prevents spindle pole separation during prometaphase, giving rise to monopolar spindles and causing mitotic arrest and induction of programmed cell death. KSP and related kinesins in other, non-human, organisms, bundle antiparallel microtubules and slide them relative to one another, thus forcing the two spindle poles apart. KSP may also mediate in anaphase B spindle elongation and focussing of microtubules at the spindle pole.

Human KSP (also termed HsEg5) has been described (Blangy, et al., Cell, 83:1159-69 (1995); Whitehead, et al., Arthritis Rheum., 39:1635-42 (1996); Galgio et al., J. Cell Biol., 135:339-414 (1996); Blangy, et al., J. Biol. Chem., 272:19418-24 (1997); Blangy, et al., Cell Motil Cytoskeleton, 40:174-82 (1998); Whitehead and Rattner, J. Cell Sci., 111:2551-61 (1998); Kaiser, et al., JBC 274:18925-31 (1999); GenBank accession numbers: X85137, NM004523 and U37426), and a fragment of the KSP gene (TRIP5) has been described (Lee, et al., Mol. Endocrinol., 9:243-54 (1995); GenBank accession number L40372). Xenopus KSP homologs (Eg5), as well as Drosophila KLP61 F/KRP1 30 have been reported.

Mitotic kinesins are attractive targets for the discovery and development of novel antimitotic chemotherapeutics. Accordingly, it is an object of the present invention to provide methods, compounds and compositions useful in the inhibition of KSP, a mitotic kinesin.

### SUMMARY OF THE INVENTION

In accordance with the objects outlined above, the present invention provides methods, compounds and compositions that can be used to treat diseases of proliferating cells. The compounds and compositions are KSP inhibitors, particularly human KSP inhibitors.

In one aspect, the invention relates to compositions useful for treating cellular proliferative disease and for inhibiting KSP kinesin. In another aspect, the invention relates to methods useful for treating cellular proliferative diseases, for treating disorders by inhibiting the activity of KSP kinesin. The methods and compositions of this invention employ the novel compounds represented by Formula I:

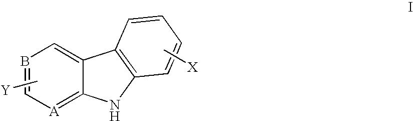

wherein:

X is selected from the group consisting of CF₃ and S(O)ₙR₁;

Y is selected from the group consisting of NR₁R₂, NR₁COR₂, NR₁CONR₂R₃, NR₁CSNR₂R₃, NR₁S(O)ₙNR₂R₃ and NR₁S(O)ₙR₂;

n is at each occurrence independently 1 or 2;

R₁, R₂ and R₃ are at each occurrence independently selected from the group consisting of hydrogen, optionally substituted alkyl, optionally substituted aryl, optionally substituted aralkyl, optionally substituted heteroaryl and optionally substituted heteroarylalkyl;

A and B are independently carbon or nitrogen; or a

pharmaceutically acceptable salt or solvate thereof.

Substituent X may occupy any of the four positions on the ring that it is located. The substituent Y may occupy any of the open positions on the ring that it is located on except for A and/or B when A and/or B is N.

In one aspect, the invention relates to methods for treating cellular proliferative diseases and other disorders that can be treated by inhibiting KSP by the administration of a therapeutically effective amount of a compound of Formula I or a pharmaceutically acceptable salt or solvate thereof. Diseases and disorders that respond to therapy with compounds of the invention include cancer, hyperplasia, restenosis, cardiac hypertrophy, immune disorders, fungal disorders and inflammation. In another aspect, the invention relates to compounds useful in inhibiting KSP kinesin. The compounds have the structures shown in Formula I. The invention also relates to a pharmaceutical composition containing a therapeutically effective amount of a compound of Formula I or a pharmaceutically acceptable salt or solvate thereof admixed with at least one pharmaceutically acceptable excipient.

In an additional aspect, the present invention provides methods of screening for compounds that will bind to a KSP kinesin, for example compounds that will displace or compete with the binding of the compounds or compositions of the invention. The methods comprise combining a labeled compound of the invention, a KSP kinesin, and at least one candidate agent and determining the binding of the candidate bioactive agent to the KSP kinesin.

In a further aspect, the invention provides methods of screening for inhibitors of KSP kinesin activity. The methods comprise combining a compound or composition of the invention, a KSP kinesin, and at least one candidate agent and determining the effect of the biological agent on the KSP kinesin activity.

### DETAILED DESCRIPTION OF THE INVENTION

### Compounds of the Present Invention

The present invention is directed to a class of novel compounds, comprising a di-substituted 9H-carbazole, 1H-pyrido[2,3-b]indole, 5H-pyrido[4,3-b]indole or 1H-pyrimido[4,5b]indole structure, that are inhibitors of mitotic kinesins. By inhibiting mitotic kinesins, but not other kinesins (e.g., transport kinesins), specific inhibition of cellular proliferation is accomplished. Thus, the present invention capitalizes on the finding that perturbation of mitotic kinesin function causes malformation or dysfunction of mitotic spindles, frequently resulting in cell cycle arrest and cell death.

An object of the present invention is to develop inhibitors of one or more mitotic kinesins, in particular KSP, for the treatment of disorders associated with cell proliferation. Traditionally, dramatic improvements in the treatment of cancer, one type of cell proliferative disorder, have been associated with identification of therapeutic agents acting through novel mechanisms. Examples of this include not only the taxane class of agents that appear to act on microtubule formation, but also the camptothecin class of topoisomerase I inhibitors. The compounds, compositions and methods described herein can differ in their selectivity and are preferably used to treat diseases of proliferating cells, including, but not limited to cancer, hyperplasias, restenosis, cardiac hypertrophy, immune disorders, fungal disorders and inflammation.

Accordingly, the present invention relates to novel compounds represented by Formula I, as well as to compositions and methods of using these compounds.

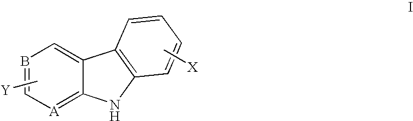

wherein:

X is selected from the group consisting of CF₃ and S(O)ₙR₁;

Y is selected from the group consisting of NR₁R₂, NR₁COR₂, NR₁CONR₂R₃, NR₁CSNR₂R₃, NR₁S(O)ₙNR₂R₃ and NR₁S(O)ₙR₂;

n is 1 or 2;

R₁, R₂ and R₃ are at each occurrence independently selected from the group consisting of hydrogen, optionally substituted alkyl, optionally substituted aryl, optionally substituted aralkyl, optionally substituted heteroaryl and optionally substituted heteroarylalkyl;

A and B are independently carbon or nitrogen; or

a pharmaceutically acceptable salt or solvate thereof.

Substituent X may occupy any of the four positions on the ring that it is located. The substituent Y may occupy any of the open positions on the ring that it is located on except for A and/or B when A and/or B is N.

In some embodiments, X is CF₃. In other embodiments, X is S(O)ₙR₁. In some embodiments, when X is S(O)ₙR₁, n is 2 and R₁ is substituted alkyl. In some embodiments, when X is S(O)ₙR₁ and n is 2, R₁ is methyl.

In some embodiments, A and B are carbon. In other embodiments, A and B are nitrogen.

In some embodiments, Y is NR₁R₂. In some embodiments when Y is NR₁R₂, R₁ and R₂ are at each occurrence independently hydrogen or substituted alkyl.

In some embodiments, when Y is NR₁R₂, R₁ and R₂ are hydrogen.

In some embodiments, Y is NR₁CONR₂R₃. In some embodiments when Y is NR₁CONR₂R₃, R₁, R₂ and R₃ are at each occurrence independently hydrogen or substituted alkyl.

In some embodiments, Y is NR₁CSNR₂R₃. In some embodiments when Y is NR₁CSNR₂R₃, R₁, R₂ and R₃ are at each occurrence independently hydrogen or substituted alkyl.

In some embodiments, Y is NR₁S(O)ₙNR₂R₃. In some embodiments when Y is NR₁S(O)ₙNR₂R₃, R₁, R₂ and R₃ are at each occurrence independently hydrogen or substituted alkyl.

In some embodiments, Y is NR₁S(O)ₙR₂. In some embodiments when Y is NR₁S(O)ₙR₂, R₁ and R₂ are at each occurrence independently hydrogen or substituted alkyl. In some embodiments when Y is NR₁S(O)ₙR₂, R₁ and R₂ are at each occurrence independently hydrogen or methyl.

In some embodiments, this invention describes compounds of formula I having the formula: a) 7-(trifluoromethyl)-1H-pyrimido[4,5b]indol-2-ylamine; 6-(trifluoromethyl)-1H-pyrimido[4,5-b]indol-2-ylamine; 6-(trifluoromethyl)-9H-carbazol-2-ylamine; 6-(trifluoromethyl)-9H-carbazol-3-ylamine; N-[7-(trifluoromethyl)-9H-carbazol-2-yl]methanesulfonamide; N-[6(trifluoromethyl)-9H-carbazol-2-yl]urea; 7-(trifluoromethyl)-9H-carbazol-3-ylamine; N-[7-(trifluoromethyl)-9H-carbazol-3-yl]urea; N-[6-(trifluoromethyl)-9H-carbazol-2-yl]sulfamide; N-[7-(trifluoromethyl)-9H-carbazol-3-yl]methanesulfonamide; N-[7-(trifluoromethyl)-9H-carbazol-2-yl]urea; N-[7-(trifluoromethyl)-9H-carbazol-3-yl]thiourea; N-[7-(trifluoromethyl)-9H-carbazol-2-yl]thiourea; 7-(trifluoromethyl)-9H-carbazol-2-ylamine; N-[7-(trifluoromethyl)-9H-carbazol-2-yl]sulfamide; or N-[7-(trifluoromethyl)-9H-carbazol-3-yl]sulfamide, or a pharmaceutically acceptable salt or solvate thereof.

In some embodiments, this invention comprises a pharmaceutically acceptable excipient and any of the previously described embodiments of formula I, or pharmaceutically acceptable salts or solvates thereof. In some embodiments, this invention comprises a pharmaceutically acceptable excipient and any of the previously described embodiments of formula I, or pharmaceutically acceptable salts or solvates thereof, and further comprises a taxane. In some embodiments, this invention comprises a pharmaceutically acceptable excipient and any of the previously described embodiments of formula I, or pharmaceutically acceptable salts or solvates thereof, and further comprises a vinca alkaloid. In some embodiments, this invention comprises a pharmaceutically acceptable excipient and any of the previously described embodiments of formula I, or pharmaceutically acceptable salts or solvates thereof, and further comprises a topoisomerase I inhibitor.

In some embodiments, this invention describes a method of inhibiting KSP which comprises contacting said kinesin with an effective amount of the compound or composition according to any one of the previously described compound or composition embodiments.

In some embodiments, this invention describes a method for the treatment of a disease of proliferating cells comprising administering to a subject in need thereof the compound or composition according to any one of the previously described compound embodiments. In some embodiments, said disease is selected from a group consisting of cancer, hyperplasias, restenosis, cardiac hypertrophy, immune disorders, fungal disorders and inflammation.

As used in the present specification, the following words and phrases are generally intended to have the meanings as set forth below, except to the extent that the context in which they are used indicates otherwise.

Alkyl is intended to include linear, branched, or cyclic hydrocarbon structures and combinations thereof. Examples of alkyl groups include methyl, ethyl, propyl, isopropyl, butyl, s- and t-butyl, pentyl, hexyl, heptyl, octyl, nonyl, decyl, undecyl, dodecyl and the like. Preferred alkyl groups range from C₂₀ and below, C₁₃ and below, C₈ and below, C₆ and below, or C₄ and below.

Cycloalkyl is a subset of alkyl and includes cyclic hydrocarbon groups of from 3 to 13 carbon atoms. Examples of cycloalkyl groups include c-propyl, c-butyl, c-pentyl, norbornyl, adamantyl and the like. In this application, alkyl refers to alkanyl, alkenyl and alkynyl residues; it is intended to include cyclohexylmethyl, vinyl, allyl, isoprenyl and the like. Alkylene is another subset of alkyl, referring to the same residues as alkyl, but having two points of attachment. Examples of alkylene include ethylene (—CH₂CH₂), propylene (—CH₂CH₂CH₂—), dimethylpropylene (—CH₂C(CH₃)₂CH₂—) and cyclohexylpropylene (—CH₂CH₂CH(C₆H₁₃)—). When an alkyl residue having a specific number of carbons is named, all geometric isomers having that number of carbons are intended to be encompassed; thus, for example, “butyl” is meant to include n-butyl, sec-butyl, isobutyl and t-butyl; “propyl” includes n-propyl and isopropyl.

Aryl means a 6-membered aromatic, a bicyclic 9- or 10-membered aromatic or a tricyclic 14- to 16-membered aromatic ring system containing. The aromatic 6- to 16-membered carbocyclic rings include such non-limiting examples e.g., phenyl, naphthyl, indanyl, tetralinyl, anthracenyl, fluorenyl, and the like.

Heteroaryl means a 5- or 6-membered heteroaromatic ring containing 1-4 heteroatoms selected singly or in combination from a group consisting of N, O, and S; a bicyclic 9- or 10-membered heteroaromatic ring system containing 1-4 heteroatoms, selected singly or in combination from a group consisting of N, O, or S; or a tricyclic 14- to 16-membered heteroaromatic ring system containing 1-4 heteroatoms selected singly or in combination from a group consisting of N, O, and S. Some non-limiting examples of 5- to 16-member heteroaromatic compounds include, e.g., imidazole, pyridinyl, indolyl, thienyl, benzopyranonyl, thiazolyl, furanyl, benzimidazolyl, quinolinyl, isoquinolinyl, quinoxalinyl, pyrimidinyl, pyrazinyl, pyrazolyl, tetrazolyl, phenothiazinyl, benzoquinolinyl, naphtho[2,3-b]thiophenyl, and the like.

Aralkyl refers to a residue in which an aryl moiety is attached to the parent structure via an alkyl residue. Examples include benzyl, phenethyl, phenylvinyl, phenylallyl and the like. Heteroaralkyl refers to a residue in which a heteroaryl moiety is attached to the parent structure via an alkyl residue. Examples include furanylmethyl, pyridinylmethyl, pyrimidinylethyl and the like.

Halogen or halo refers to fluorine, chlorine, bromine or iodine. Fluorine, chlorine and bromine are preferred. Dihaloaryl, dihaloalkyl, trihaloaryl etc. refer to aryl and alkyl substituted with a plurality of halogens, but not necessarily a plurality of the same halogen; thus 4-chloro-3-fluorophenyl is within the scope of dihaloaryl.

Substituted-alkyl, aryl, and heteroaryl, which includes the substituted alkyl, aryl and heteroaryl moieties of any group containing an optionally substituted alkyl, aryl and heteroaryl moiety (e.g., aralkyl and heteroaralkyl), refer respectively to alkyl, aryl, and heteroaryl wherein one or more (up to about 5, preferably up to about 3) hydrogen atoms are replaced by a substituent, wherein each substituent is independently selected from the group:
—R^{a}, —OR^{b}, —O(C₁-C₂ alkyl)O— (as an aryl substituent), —SR^{b}, NR^{b}R^{c}, —C(═NR^{c})—NR^{b}R^{c}, halogen, cyano, nitro, —COR^{b}, —CO₂R^{b}, —CONR^{b}R^{c}, —OCOR^{b}, —OCO₂R^{b}, OCONR^{b}R^{c}, —NR^{c}COR^{b}, —NR^{c}CO₂R^{b}, —NR^{c}CONR^{b}R^{c}, CO₂R^{b}, —CONR^{b}R^{c}, —NR^{c}COR^{b}, —SOR^{a}, —SO₂R^{a}, —SO₂NR^{b}R^{c}, and —NR^{c}SO₂R^{a}, where R^{a} is at each occurrence independently optionally substituted C₁-C₈ alkyl, optionally substituted aryl, optionally substituted heteroaryl, optionally substituted aryl-C₁-C₄ alkyl-, or optionally substituted heteroaryl-C₁-C₄ alkyl- group,
R^{b} is at each occurrence independently H, optionally substituted C₁-C₆ alkyl, optionally substituted aryl, optionally substituted heteroaryl, optionally substituted aryl-C₁-C₄ alkyl-, or optionally substituted heteroaryl-C₁-C₄ alkyl-group;
R^{c} is hydrogen or C₁-C₄ alkyl;
where each optionally substituted R^{a} group and R^{b} group is independently unsubstituted or substituted with one or more substituents independently selected from C₁-C₄ alkyl, aryl, heteroaryl, aryl-C₁-C₄ alkyl-, heteroaryl-C₁-C₄ alkyl-, C₁-C₄ haloalkyl, —OC₁-C₄ alkyl, —OC₁-C₄ alkylphenyl, —C₁-C₄ alkyl-OH, —OC₁-C₄ haloalkyl, halogen, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo (as a substitutent for heteroaryl), —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ phenyl, —C(O)C₁-C₄ haloalkyl, —OC(O)C₁-C₄ alkyl, —SO₂(C₁-C₄ alkyl), —SO₂(phenyl), —SO₂(C₁-C₄ haloalkyl), —SO₂NH₂, —SO₂NH(C₁-C₄ alkyl), —SO₂NH(phenyl), —NHSO₂(C₁-C₄ alkyl), —NHSO₂(phenyl), and —NHSO₂(C₁-C₄ haloalkyl).

It will be understood that “optional” or “optionally” means that the subsequently described event or circumstance may or may not occur, and that the description includes instances where said event or circumstance occurs and instances in which it does not. It will be understood that when a group or moiety is “optionally substituted,” the group or moiety may be unsubstituted or may be substituted by one or more of the substituents defined herein, where each substituent is selected independently. It will be further understood by those skilled in the art with respect to any groups containing one or more substituents that such groups are not intended to introduce any substituent or substitution patterns that are sterically impractical and/or synthetically non-feasible and/or inherently unstable.

Pharmaceutically acceptable acid addition salt refers to those salts that retain at least some of the biological effectiveness of the free bases, and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, benzenesulfonic acid, p-toluenesulfonic acid, salicylic acid and the like. The acids used may be monoprotic, diprotic, or polyprotic (including polymeric acids), and thus the stoichiometry for di- or polyprotic acid derived salts can be adjusted through a range according to what is desired.

Pharmaceutically acceptable base addition salt refers to those salts that retain at least some of the biological effectiveness of a free acid, and that are not biologically or otherwise undesirable, formed with inorganic or organic bases including those containing sodium, potassium, lithium, ammonium, calcium, magnesium, iron, zinc, copper, manganese, aluminum, and the like. Particularly preferred base addition salt products are ammonium, potassium, sodium, calcium, and magnesium salts. Salts derived from pharmaceutically acceptable organic non-toxic bases include salts of primary, secondary, and tertiary amines. The bases used may also be monobasic, dibasic, or polybasic (including base resins), and thus the stoichiometry for di- or polybase derived salts of the final product can be adjusted through a range according to what is desired.

Pharmaceutically acceptable solvates refer to the complex formed by the interaction of a solvent and a compound of Formula I or a pharmaceutically acceptable salt or derivative thereof. Suitable solvates are those formed with pharmaceutically acceptable solvents, including hydrates (i.e., wherein the solvent is water). It will be understood that phrases such as “a compound of Formula I or a pharmaceutically acceptable salt or solvate thereof” are intended to encompass the compound of Formula I, a pharmaceutically acceptable salt of the compound, a solvate of the compound and a solvate of a pharmaceutically acceptable salt of the compound. Certain embodiments of the invention described herein may contain one or more asymmetric centers and may thus give rise to enantiomers, diastereomers, and other stereoisomeric forms that may be defined, in terms of absolute stereochemistry, as (R)- or (S)-. The present invention is meant to include all such possible isomers, including racemic mixtures, optically pure forms and intermediate mixtures. Optically active (R)- and (S)-isomers may be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers. Likewise, all tautomeric forms are also intended to be included.

When desired, the R- and S-isomers may be resolved by methods known to those skilled in the art, for example by formation of diastereoisomeric salts or complexes which may be separated, for example, by crystallisation; via formation of diastereoisomeric derivatives which may be separated, for example, by crystallisation, gas-liquid or liquid chromatography; selective reaction of one enantiomer with an enantiomer-specific reagent, for example enzymatic oxidation or reduction, followed by separation of the modified and unmodified enantiomers; or gas-liquid or liquid chromatography in a chiral environment, for example on a chiral support, such as silica with a bound chiral ligand or in the presence of a chiral solvent. It will be appreciated that where the desired enantiomer is converted into another chemical entity by one of the separation procedures described above, a further step may be required to liberate the desired enantiomeric form. Alternatively, a specific enantiomer may be synthesized by asymmetric synthesis using optically active reagents, substrates, catalysts or solvents, or by converting on enantiomer to the other by asymmetric transformation.

The compounds and compositions of the invention find use in a variety of applications. As will be appreciated by those in the art, mitosis may be altered in a variety of ways; that is, one can affect mitosis either by increasing or decreasing the activity of a component in the mitotic pathway. Stated differently, mitosis may be affected (e.g., disrupted) by disturbing equilibrium, either by inhibiting or activating certain components. Similar approaches may be used to alter meiosis.

In a preferred embodiment, the compounds and compositions of the invention are used to inhibit mitotic spindle formation, thus causing prolonged cell cycle arrest in mitosis. By “inhibit” herein is meant decreasing spindle formation. By “mitotic spindle formation” herein is meant organization of microtubules into bipolar structures by mitotic kinesins. By “mitotic spindle dysfunction” herein is meant mitotic arrest and monopolar spindle formation.

The compositions of the invention are useful to bind to and/or inhibit the activity of a mitotic kinesin, KSP. In a preferred embodiment, the KSP is human KSP, although KSP kinesins from other organisms may also be used. In this context, inhibit means decreasing spindle pole separation, causing malformation, i.e., splaying, of mitotic spindle poles, or otherwise causing morphological perturbation of the mitotic spindle. Also included within the definition of KSP for these purposes are variants and/or fragments of KSP. See U.S. Pat. No. 6,414,121, hereby incorporated by reference in its entirety. In addition, other mitotic kinesins may be used in the present invention. Preferred compounds of the invention demonstrate at least some specificity for KSP. For assay of activity, generally either KSP or a compound according to the invention is non-diffusably bound to an insoluble support having isolated sample receiving areas (e.g., a microtiter plate, an array, etc.). The insoluble support may be made of any composition to which the compositions can be bound, is readily separated from soluble material, and is otherwise compatible with the overall method of screening. The surface of such supports may be solid or porous and of any convenient shape. Examples of suitable insoluble supports include microtiter plates, arrays, membranes and beads. These are typically made of glass, plastic (e.g., polystyrene), polysaccharides, nylon or nitrocellulose, Teflon™, etc. Microtiter plates and arrays are especially convenient because a large number of assays can be carried out simultaneously, using small amounts of reagents and samples. The particular manner of binding of the composition is not crucial so long as it is compatible with the reagents and overall methods of the invention, maintains the activity of the composition and is nondiffusable. Preferred methods of binding include the use of antibodies (which do not sterically block either the ligand binding site or activation sequence when the protein is bound to the support), direct binding to “sticky” or ionic supports, chemical crosslinking, the synthesis of the protein or agent on the surface, etc. Following binding of the protein or agent, excess unbound material is removed by washing. The sample receiving areas may then be blocked through incubation with bovine serum albumin (BSA), casein or other innocuous protein or other moiety.

The mitotic agents of the invention may be used on their own to inhibit the activity of a mitotic kinesin, particularly KSP. In this embodiment, the mitotic agents of the invention are combined with KSP and the activity of KSP is assayed. Kinesin activity is known in the art and includes one or more kinesin activities. Kinesin activities include the ability to affect ATP hydrolysis; microtubule binding; gliding and polymerization/depolymerization (effects on microtubule dynamics); binding to other proteins of the spindle; binding to proteins involved in cell-cycle control; serving as a substrate to other enzymes; such as kinases or proteases; and specific kinesin cellular activities such as spindle pole separation.

Methods of performing motility assays are well known to those of skill in the art. (See e.g., Hall, et al. (1996), Biophys. J., 71: 3467-3476, Turner et al., 1996, Anal. Biochem. 242 (1):20-5; Gittes et al., 1996, Biophys. J. 70(I): 418-29; Shirakawa et al., 1995, J. Exp. Biol. 198: 1809-15; Winkelmann et al., 1995, Biophys. J. 68: 2444-53; Winkelmann et al., 1995, Biophys. J. 68: 72S.)

Methods known in the art for determining ATPase hydrolysis activity also can be used. Preferably, solution based assays are utilized. U.S. application Ser. No. 09/314,464, filed May 18, 1999, hereby incorporated by reference in its entirety, describes such assays. Alternatively, conventional methods are used. For example, Pᵢ release from kinesin can be quantified. In one preferred embodiment, the ATPase hydrolysis activity assay utilizes 0.3 M PCA (perchloric acid) and malachite green reagent (8.27 mM sodium molybdate II, 0.33 mM malachite green oxalate, and 0.8 mM Triton X-100). To perform the assay, 10 μL of reaction is quenched in 90 μL of cold 0.3 M PCA. Phosphate standards are used so data can be converted to mM inorganic phosphate released. When all reactions and standards have been quenched in PCA, 100 μL of malachite green reagent is added to the relevant wells in e.g., a microtiter plate. The mixture is developed for 10-15 minutes and the plate is read at an absorbance of 650 nm. If phosphate standards were used, absorbance readings can be converted to mM Pᵢ and plotted over time. Additionally, ATPase assays known in the art include the luciferase assay.

ATPase activity of kinesin motor domains also can be used to monitor the effects of inhibitory agents. In one embodiment ATPase assays of kinesin are performed in the absence of microtubules. In another embodiment, the ATPase assays are performed in the presence of microtubules. Different types of inhibiting agents can be detected in the above assays. In some embodiment, the effect of the agents on kinesin ATPase can be decreased by increasing the concentrations of ATP, microtubules or both. Agents that inhibit the biochemical activity of KSP in vitro may then be screened in vivo. Methods for such agents in vivo include assays of cell cycle distribution, cell viability, or the presence, morphology, activity, distribution, or amount of mitotic spindles. Methods for monitoring cell cycle distribution of a cell population, for example, by flow cytometry, are well known to those skilled in the art, as are methods for determining cell viability. See for example, U.S. patent application “Methods of Screening for Modulators of Cell Proliferation and Methods of Diagnosing Cell Proliferation States,” U.S. Pat. No. 6,414,121, hereby incorporated by reference in its entirety. In addition to the assays described above, microscopic methods for monitoring spindle formation and malformation are well known to those of skill in the art (see, e.g., Whitehead and Rattner (1998), J. Cell Sci. 111:2551-61; Galgio et al, (1996) J. Cell biol., 135:399-414).

The compositions of the invention inhibit the KSP kinesin. One measure of inhibition is IC₅₀, defined as the concentration of the composition at which the activity of KSP is decreased by fifty percent relative to a control. Preferred compositions have IC_{═}'s of less than about 1 mM, with preferred embodiments having IC₅₀'s of less than about 100 μM, with more preferred embodiments having IC₅₀'s of less than about 10 μM, with particularly preferred embodiments having IC₅₀'s of less than about 1 μM, and especially preferred embodiments having IC₅₀'s of less than about 100 nM, and with the most preferred embodiments having IC₅₀'s of less than about 10 nM. Measurement of IC₅₀ is done using an ATPase assay.

Another measure of inhibition is Kᵢ. For compounds with IC₅₀'s less than 1 μM, the Kᵢ or K_{d} is defined as the dissociation rate constant for the interaction of the compounds described herein with KSP. Preferred compounds have Kᵢ's of less than about 100 μM, with preferred embodiments having Kᵢ's of less than about 10 μM, and particularly preferred embodiments having Kᵢ's of less than about 1 μM and especially preferred embodiments having Kᵢ's of less than about 100 nM, and with the most preferred embodiments having Kᵢ's of less than about 10 nM. The K for a compound is determined from the IC₅₀ based on three assumptions. First, only one compound molecule binds to the enzyme and there is no cooperativity. Second, the concentrations of active enzyme and the compound tested are known (i.e., there are no significant amounts of impurities or inactive forms in the preparations). Third, the enzymatic rate of the enzyme-inhibitor complex is zero. The rate (i.e., compound concentration) data are fitted to the equation:

$$V = {V}_{max} {E}_{0} [ I - \frac{( {E}_{0} + {I}_{0} + Kd ) - \sqrt{{( {E}_{0} + {I}_{0} + Kd )}^{2} - 4 {E}_{0} {I}_{0}}}{2 {E}_{0}} ] .$$

where V is the observed rate, Vₘₐₓ is the rate of the free enzyme, I₀ is the inhibitor concentration, E₀ is the enzyme concentration, and K_{d} is the dissociation constant of the enzyme-inhibitor complex.

Another measure of inhibition is the cell IC₅₀, defined as the concentration of the compound that results in a decrease in the rate of cell growth by fifty percent. Preferred compounds have cell IC₅₀'s of less than about 1 mM. The level of preferability of embodiments is a function of their cell IC₅₀: those having cell IC₅₀'s of less than about 20 μM are more preferred; those having cell IC₅₀'s of 10 μM more so; those having cell IC₅₀'s of less than about 1 μM more so; those having cell IC₅₀'s of 100 nM even more so. Measurement of cell IC₅₀'s is done using a cell proliferation assay.

The compounds and compositions of the invention are used to treat cellular proliferation diseases. Disease states which can be treated by the methods, compounds and compositions provided herein include, but are not limited to, cancer, autoimmune disease, fungal disorders, arthritis, graft rejection, inflammatory bowel disease, proliferation induced after medical procedures, including, but not limited to, surgery, angioplasty, and the like. It is appreciated that in some cases the cells may not be in a hyper or hypo proliferation state (abnormal state) and still require treatment. For example, during wound healing, the cells may be proliferating “normally”, but proliferation enhancement maybe desired. Thus, in one embodiment, the invention herein includes application to cells or individuals afflicted or impending affliction with any one of these disorders or states.

The compositions and methods provided herein are particularly deemed useful for the treatment of cancer including solid tumors such as skin, breast, brain, cervical carcinomas, testicular carcinomas, etc. More particularly, cancers that may be treated by the compositions and methods of the invention include, but are not limited to:

Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma; leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Karposi's sarcoma, moles dysplastic nevi, lipoma, angioma, dermatofibroma, keloids, psoriasis; and Adrenal glands: neuroblastoma. Thus, the term “cancerous cell” as provided herein, includes a cell afflicted by any one of the above identified conditions.

Accordingly, the compositions of the invention are administered to cells. By “administered” herein is meant administration of a therapeutically effective dose of the mitotic agents of the invention to a cell either in cell culture or in a patient. By “therapeutically effective dose” herein is meant a dose that produces the effects for which it is administered. The exact dose will depend on the purpose of the treatment, and will be ascertainable by one skilled in the art using known techniques. As is known in the art, adjustments for systemic versus localized delivery, age, body weight, general health, sex, diet, time of administration, drug interaction and the severity of the condition may be necessary, and will be ascertainable with routine experimentation by those skilled in the art. By “cells” herein is meant almost any cell in which mitosis or meiosis can be altered.

A “patient” for the purposes of the present invention includes both humans and other animals, particularly mammals, and other organisms. Thus the methods are applicable to both human therapy and veterinary applications. In the preferred embodiment the patient is a mammal, and in the most preferred embodiment the patient is human.

Mitotic agents having the desired pharmacological activity may be administered in a physiologically acceptable carrier to a patient, as described herein. Depending upon the manner of introduction, the compounds may be formulated in a variety of ways as discussed below. The concentration of therapeutically active compound in the formulation may vary from about 0.1-100 wt. %. The agents may be administered alone or in combination with other treatments, i.e., radiation, or other therapeutic agents, such as the taxane class of agents that appear to act on microtubule formation or the camptothecin class of topoisomerase I inhibitors. When so used, other therapeutic agents can be administered before, concurrently (whether in separate dosage forms or in a combined dosage form), or after administration of an active agent of the present invention.

Pharmaceutical formulations include a compound of Formula I or a pharmaceutically acceptable salt or solvate thereof, and one or more pharmaceutically acceptable excipients. As is known in the art, pharmaceutical excipients are secondary ingredients that function to enable or enhance the delivery of a drug or medicine in a variety of dosage forms (e.g., oral forms such as tablets, capsules, and liquids; topical forms such as dermal, ophthalmic, and otic forms; suppositories; injectables; respiratory forms and the like). Pharmaceutical excipients include inert or inactive ingredients, synergists or chemicals that substantively contribute to the medicinal effects of the active ingredient. For example, pharmaceutical excipients may function to improve flow characteristics, product uniformity, stability, taste, or appearance, to ease handling and administration of dose, for convenience of use, or to control bioavailability. While pharmaceutical excipients are commonly described as being inert or inactive, it is appreciated in the art that there is a relationship between the properties of the pharmaceutical excipients and the dosage forms containing them. Pharmaceutical excipients suitable for use as carriers or diluents are well known in the art, and maybe used in a variety of formulations. See, e.g., Remington's Pharmaceutical Sciences, 18th Edition, A. R. Gennaro, Editor, Mack Publishing Company (1990); Remington: The Science and Practice of Pharmacy, 20th Edition, A. R. Gennaro, Editor, Lippincott Williams & Wilkins (2000); Handbook of Pharmaceutical Excipients, 3rd Edition, A. H. Kibbe, Editor, American Pharmaceutical Association, and Pharmaceutical Press (2000); and Handbook of Pharmaceutical Additives, compiled by Michael and Irene Ash, Gower (1995). The concentration of a therapeutically active agent in a formulation can vary widely, from about 0.1 to 99.9 wt. %, depending on the nature of the formulation.

Oral solid dosage forms such as tablets will typically comprise one or more pharmaceutical excipients, which may for example help impart satisfactory processing and compression characteristics, or provide additional desirable physical characteristics to the tablet. Such pharmaceutical excipients may be selected from diluents, binders, glidants, lubricants, disintegrants, colorants, flavorants, sweetening agents, polymers, waxes or other solubility modulating agents.

Dosage forms for parenteral administration will generally comprise fluids, particularly intravenous fluids, i.e., sterile solutions of simple chemicals such as sugars, amino acids or electrolytes, which can be easily carried by the circulatory system and assimilated. Such fluids are typically prepared with water for injection USP. Fluids used commonly for intravenous (IV) use are disclosed in Remington, the Science and Practice of Pharmacy [full citation previously provided], and include:
alcohol, e.g., 5% alcohol (e.g., in dextrose and water (“D/W”) or D/W in normal saline solution (“NSS”), including in 5% dextrose and water (“D5/W”), or D5/W in NSS);
synthetic amino acid such as Aminosyn, FreAmine, Travasol, e.g., 3.5 or 7; 8.5; 3.5, 5.5 or 8.5%, respectively;
ammonium chloride e.g., 2.14%;
dextran 40, in NSS e.g., 10% or in D5/W e.g., 10%;
dextran 70, in NSS e.g., 6% or in D5/W e.g., 6%;
dextrose (glucose, D5/W) e.g. 2.5-50%;
dextrose and sodium chloride e.g., 520% dextrose and 0.22-0.9% NaCl;
lactated Ringer's (Hartmann's) e.g., NaCl 0.6%, KCl 0.03%, CaCl₂ 0.02%;
lactate 0.3%;
mannitol e.g., 5%, optionally in combination with dextrose e.g., 10% or NaCl e.g., 15 or 20%;
multiple electrolyte solutions with varying combinations of electrolytes, dextrose, fructose, invert sugar Ringer's e.g., NaCl 0.86%, KCl 0.03%, CaCL2 0.033%;
sodium bicarbonate e.g., 5%;
sodium chloride e.g., 0.45, 0.9, 3, or 5%;
sodium lactate e.g., ⅙ M; and
sterile water for injection.

The pH of such fluids may vary, and will typically be from 3.5 to 8 as known in the art.

The pharmaceutical compositions can be prepared in various forms, such as granules, tablets, pills, suppositories, capsules, suspensions, salves, lotions and the like. Pharmaceutical grade organic or inorganic carriers and/or diluents suitable for oral and topical use can be used to make up compositions containing the therapeutically-active compounds. Diluents known to the art include aqueous media, vegetable and animal oils and fats. Stabilizing agents; wetting and emulsifying agents, salts for varying the osmotic pressure or buffers for securing an adequate pH value, and skin penetration enhancers can be used as auxiliary agents. The pharmaceutical compositions may also include one or more of the following: carrier proteins such as serum albumin; buffers; fillers such as microcrystalline cellulose, lactose, corn and other starches; binding agents; sweeteners and other flavoring agents; coloring agents; and polyethylene glycol. Additives are well known in the art, and are used in a variety of formulations.

The administration of the agents of the present invention can be done in a variety of ways as discussed above, including, but not limited to, orally, subcutaneously, intravenously, intranasally, transdermally, intraperitoneally, intramuscularly, intrapulmonary, vaginally, rectally, or intraocularly. In some instances, for example, in the treatment of wounds and inflammation, the agents may be directly applied as a solution or spray.

To employ the compounds of the invention in a method of screening for compounds that bind to KSP kinesin, the KSP is bound to a support, and a compound of the invention (which is an anti-mitotic agent) is added to the assay. Alternatively, the compound of the invention is bound to the support and KSP is added. Classes of compounds among which novel binding agents may be sought include specific antibodies, non-natural binding agents identified in screens of chemical libraries, peptide analogs, etc. Of particular interest are screening assays for candidate agents that have a low toxicity for human cells. A wide variety of assays may be used for this purpose, including labeled in vitro protein-protein binding assays, electrophoretic mobility shift assays, immunoassays for protein binding, functional assays (phosphorylation assays, etc.) and the like.

The determination of the binding of the anti-mitotic agent to KSP may be done in a number of ways. In a preferred embodiment, the anti-mitotic agent (the compound of the invention) is labeled, for example, with a fluorescent or radioactive moiety and binding determined directly. For example, this may be done by attaching all or a portion of KSP to a solid support, adding a labeled anti-mitotic agent (for example a compound of the invention in which at least one atom has been replaced by a detectable isotope), washing off excess reagent, and determining whether the amount of the label is that present on the solid support. Various blocking and washing steps may be utilized as is known in the art.

By “labeled” herein is meant that the compound is either directly or indirectly labeled with a label which provides a detectable signal, e.g., radioisotope, fluorescent tag, enzyme, antibodies, particles such as magnetic particles, chemiluminescent tag, or specific binding molecules, etc. Specific binding molecules include pairs, such as biotin and streptavidin, digoxin and antidigoxin etc. For the specific binding members, the complementary member would normally be labeled with a molecule which provides for detection, in accordance with known procedures, as outlined above. The label can directly or indirectly provide a detectable signal.

In some embodiments, only one of the components is labeled. For example, the kinesin proteins may be labeled at tyrosine positions using ¹²⁵I, or with fluorophores. Alternatively, more than one component may be labeled with different labels; using ¹²⁵I for the proteins, for example, and a fluorophor for the anti-mitotic agents.

The compounds of the invention may also be used as competitors to screen for additional drug candidates. “Candidate bioactive agent” or “drug candidate” or grammatical equivalents as used herein describe any molecule, e.g., protein, oligopeptide, small organic molecule, polysaccharide, polynucleotide, etc., to be tested for bioactivity. They may be capable of directly or indirectly altering the cellular proliferation phenotype or the expression of a cellular proliferation sequence, including both nucleic acid sequences and protein sequences. In other cases, alteration of cellular proliferation protein binding and/or activity is screened. Screens of this sort may be performed either in the presence or absence of microtubules. In the case where protein binding or activity is screened, preferred embodiments exclude molecules already known to bind to that particular protein, for example, polymer structures such as microtubules, and energy sources such as ATP. Preferred embodiments of assays herein include candidate agents which do not bind the cellular proliferation protein in its endogenous native state termed herein as “exogenous” agents. In another preferred embodiment, exogenous agents further exclude antibodies to KSP.

Candidate agents can encompass numerous chemical classes, though typically they are organic molecules, preferably small organic compounds having a molecular weight of more than 100 and less than about 2,500 daltons. Candidate agents comprise functional groups necessary for structural interaction with proteins, particularly hydrogen bonding and lipophilic binding, and typically include at least an amine, carbonyl, hydroxyl, ether, or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Particularly preferred are peptides. Candidate agents are obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fungal, plant and animal extracts are available or readily produced. Additionally, natural or synthetically produced libraries and compounds are readily modified through conventional chemical, physical and biochemical means. Known pharmacological agents may be subjected to directed or random chemical modifications, such as acylation, alkylation, esterification, amidification to produce structural analogs.

Competitive screening assays may be done by combining KSP and a drug candidate in a first sample. A second sample comprises a anti-mitotic agent, KSP and a drug candidate. This may be performed in either the presence or absence of microtubules. The binding of the drug candidate is determined for both samples, and a change, or difference in binding between the two samples indicates the presence of an agent capable of binding to KSP and potentially modulating its activity. That is, if the binding of the drug candidate is different in the second sample relative to the first sample, the drug candidate is capable of binding to KSP.

In a preferred embodiment, the binding of the candidate agent is determined through the use of competitive binding assays. In this embodiment, the competitor is a binding moiety known to bind to KSP, such as an antibody, peptide, binding partner, ligand, etc. Under certain circumstances, there may be competitive binding as between the candidate agent and the binding moiety, with the binding moiety displacing the candidate agent.

In one embodiment, the candidate agent is labeled. Either the candidate agent, or the competitor, or both, is added first to KSP for a time sufficient to allow binding, if present. Incubations may be performed at any temperature which facilitates optimal activity, typically between 4 and 40° C.

Incubation periods are selected for optimum activity, but may also be optimized to facilitate rapid high throughput screening. Typically between 0.1 and 1 hour will be sufficient. Excess reagent is generally removed or washed away. The second component is then added, and the presence or absence of the labeled component is followed, to indicate binding.

In a preferred embodiment, the competitor is added first, followed by the candidate agent. Displacement of the competitor is an indication the candidate agent is binding to KSP and thus is capable of binding to, and potentially modulating, the activity of KSP. In this embodiment, either component can be labeled. Thus, for example, if the competitor is labeled, the presence of label in the wash solution indicates displacement by the agent. Alternatively, if the candidate agent is labeled, the presence of the label on the support indicates displacement.

In an alternative embodiment, the candidate agent is added first, with incubation and washing, followed by the competitor. The absence of binding by the competitor may indicate the candidate agent is bound to KSP with a higher affinity. Thus, if the candidate agent is labeled, the presence of the label on the support, coupled with a lack of competitor binding, may indicate the candidate agent is capable of binding to KSP.

It may be of value to identify the binding site of KSP. This can be done in a variety of ways. In one embodiment, once KSP has been identified as binding to the anti-mitotic agent, KSP is fragmented or modified and the assays repeated to identify the necessary components for binding.

Modulation is tested by screening for candidate agents capable of modulating the activity of KSP comprising the steps of combining a candidate agent with KSP, as above, and determining an alteration in the biological activity of KSP. Thus, in this embodiment, the candidate agent should both bind to KSP (although this may not be necessary), and alter its biological or biochemical activity as defined herein. The methods include both in vitro screening methods and in vivo screening of cells for alterations in cell cycle distribution, cell viability, or for the presence, morphology, activity, distribution, or amount of mitotic spindles, as are generally outlined above. Alternatively, differential screening may be used to identify drug candidates that bind to the native KSP, but cannot bind to modified KSP.

Positive controls and negative controls may be used in the assays. Preferably all control and test samples are performed in at least triplicate to obtain statistically significant results. Incubation of all samples is for a time sufficient for the binding of the agent to the protein. Following incubation, all samples are washed free of non-specifically bound material and the amount of bound, generally labeled agent determined. For example, where a radiolabel is employed, the samples may be counted in a scintillation counter to determine the amount of bound compound.

A variety of other reagents may be included in the screening assays. These include reagents like salts, neutral proteins, e.g., albumin, detergents, etc which may be used to facilitate optimal protein-protein binding and/or reduce non-specific or background interactions. Also reagents that otherwise improve the efficiency of the assay, such as protease inhibitors, nuclease inhibitors, anti-microbial agents, etc., may be used. The mixture of components may be added in any order that provides for the requisite binding.

The following examples serve to more fully describe the manner of using the above-described invention, as well as to set forth the best modes contemplated for carrying out various aspects of the invention. It is understood that these examples in no way serve to limit the true scope of this invention, but rather are presented for illustrative purposes. All references cited herein are incorporated by reference in their entirety. The terms “solvent”, “inert organic solvent” or “inert solvent” mean a solvent inert under the conditions of the reaction being described in conjunction therewith [including, for example, benzene, toluene, acetonitrile, tetrahydrofuran (“THF”), dimethylformamide (“DMF”), chloroform, methylene chloride (or dichloromethane), diethyl ether, methanol, pyridine and the like]. Unless specified to the contrary, the solvents used in the reactions of the present invention are inert organic solvents. The term “q.s.” means adding a quantity sufficient to achieve a stated function, e.g., to bring a solution to the desired volume (i.e., 100%).

Isolation and purification of the compounds and intermediates described herein can be affected, if desired, by any suitable separation or purification procedure such as, for example, filtration, extraction, crystallization, column chromatography, thin-layer chromatography or thick-layer chromatography, or a combination of these procedures. Specific illustrations of suitable separation and isolation procedures can be had by reference to the examples hereinbelow. However, other equivalent separation or isolation procedures can, of course, also be used.

### EXAMPLES

The following abbreviations and terms have the indicated meanings throughout: Ac represents acetyl, BNB represents 4-bromomethyl-3-nitrobenzoic acid, Boc represents t-butyloxy carbonyl, Bu represents butyl, c- represents cyclo, CBZ represents carbobenzoxy represents benzyloxycarbonyl, DBU represents diazabicyclo[5.4.0]undec-7-ene, DCM represents dichloromethane represents methylene chloride represents CH₂Cl₂, DCE represents dichloroethylene, DEAD represents diethyl azodicarboxylate, DIC represents diisopropylcarbodiimide, DIEA represents N,N-diisopropylethyl amine, DMAP represents 4-N,N-dimethylaminopyridine, DMF represents N,N-dimethylformamide, DMSO represents dimethyl sulfoxide, DVB represents 1,4-divinylbenzene, EEDQ represents 2-ethoxy-1-ethoxycarbonyl-1,2-dihydroquinoline, Et represents ethyl, Fmoc represents 9 fluorenylmethoxycarbonyl, GC represents gas chromatography, HATU represents O-(7-Azabenzotriazol-1-yl)-1,1,3,3-tetramethyluronium, hexafluorophosphate, HMDS represents hexamethyldisilazane, HOAc represents acetic acid, HOBt represents hydroxybenzotriazole, Me represents methyl, mesyl represents methanesulfonyl, MTBE represents methyl t-butyl ether, NMO represents N-methylmorpholine oxide, PEG represents polyethylene glycol, Ph represents phenyl, PhOH represents phenol, PfP represents pentafluorophenol, PPTS represents pyridinium p-toluenesulfonate, Py represents pyridine, PyBroP represents bromo-trs-pyrrolidino-phosphonium hexafluorophosphate, rt or RT represent room temperature, sat'd represents saturated, s- represents secondary, t- represents tertiary, TBDMS represents t-butyldimethylsilyl, TES represents triethylsilyl, TFA represents trifluoroacetic acid, THF represents tetrahydrofuran, TMOF represents trimethyl orthoformate, TMS represents trimethylsilyl, tosyl represents p-toluenesulfonyl, and Trt represents triphenylmethyl. The following examples of the invention were prepared as shown in schemes 1-3 and as described below.

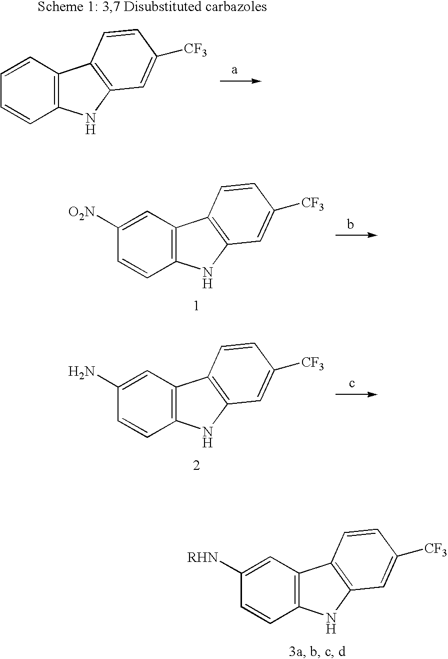

Conditions: a) HNO₃, HOAc; b) Zn, HOAc; c) For R=CH₃SO₂— (3a) CH₃SO₂Cl, pyridine, CH₂Cl₂; For R=H₂NCO— (3b) KOCN, HOAc; For R=H₂NCS— (3c) benzoyl isothiocyanate, acetone; 5% NaOH; For R=H₂NSO₂— (3d) H₂NSO₂Cl, pyridine, CH₃CN, dioxane.

### Example 1

### 7-(trifluoromethyl)-9H-carbazol-3-ylamine

2-(Trifluoromethyl)-9H-carbazole was synthesized according to the procedure of E. Forbes, M. Stacey, J. Tatlow and R. Wragg; Tetrahedron, 1960, 8, 67-72.

### 1a. 6-Nitro-2-(trifluoromethyl)-9H-carbazole

To a suspension of 2-(trifluoromethyl)-9H-carbazole (1.5 g, 6.4 mMol) in HOAc (40 mL) was added, dropwise with stirring at RT, 90% HNO₃ (340 uL, 7.2 mMol). The reaction was heated to 60° C. for 4 h (the reaction gradually became clear), cooled to RT then concentrated under vacuum. Purification by flash chromatography on silica gel (20 to 30% EtOAc in Hexanes) gave the title compound (1.11 g, 62%) as a yellow solid: MS (ES) m/e 280.8 (M+H)⁺; ¹H NMR (400 MHz, d₆-DMSO) □ 12.37 (s, 1H), 9.31 (d, J=2.3 Hz, 1H), 8.62 (d, J=8.2 Hz, 1H), 8.37 (dd, J=2.3, 9.0 Hz, 1H), 7.94 (s, 1H), 7.75 (d, J=9.0 Hz, 1H), 7.60 (dd, J=1.0, 8.2 Hz, 1H).

A faster running regioisomeric 1-nitro-7-(trifluoromethyl)-9H-carbazole (0.59 g, 33%) was also isolated as a yellow solid: MS (ES) m/e 281.0 (M+H)⁺; ¹H NMR (400 MHz, CDCl₃) □10.22 (br s, 1H), 8.46 (d, J=7.6 Hz, 1H), 8.45 (d, J=8.2 Hz, 1H), 8.23 (d, J=8.2 Hz, 1H), 7.91 (s, 1H), 7.63 (d, J=8.2 Hz, 1H), 7.42 (app. t, J=8.0 Hz, 1H).

### 1b. 7-(trifluoromethyl)-9H-carbazol-3-ylamine

To 6-nitro-2-(trifluoromethyl)-9H-carbazole (1.1 g, 3.9 mMol) suspended in HOAc (50 mL) was added with stirring at RT zinc powder (1.8 g, 27.5 mMol) portionwise. The reaction was stirred for 18 h, filtered through a pad of Celite®, rinsed with HOAc and concentrated under vacuum. The purplish solid which remained was taken up in EtOAc, washed with 1 N Na₂CO₃, brine, dried (Na₂SO₄) and evaporated to dryness. Purification by flash chromatography on silica gel (50 to 70% EtOAc in hexanes), followed by trituration with hexane, filtration and drying under vacuum gave the title compound (745 mg, 76%) as a beige solid: MS (ES) m/e 251.2 (M+H)⁺; ¹H NMR (400 MHz, d₆-DMSO) □ 11.16 (s, 1H), 8.11 (d, J=8.1 Hz, 1H), 7.69 (s, 1H), 7.33 (dd, 1H), 7.31 (s, 1H), 7.30 (d, J=8.5 Hz, 1H), 6.88 (dd, J=2.0, 8.6 Hz, 1H), 4.92 (br s, 2H).

### Example 2

### N-[7-(Trifluoromethyl)-9H-carbazol-3-yl]methanesulfonamide

To a stirred solution of 7-(trifluoromethyl)-9H-carbazol-3-amine from Example 1b above (120 mg, 0.48 mMol) in pyridine (5 mL) was added methanesulfonyl chloride (38 uL, 0.49 mMol). The reaction was stirred at RT for 18 h then concentrated under vacuum. The residue was taken up in EtOAc, washed with 1 N HCl, brine, dried (MgSO₄) and evaporated to dryness. Purification by flash chromatography on silica gel (10 to 20% EtOAc in hexanes), followed by trituration with hexane, filtration and drying under vacuum gave the title product (128.9 mg, 82%) as an off-white solid: MS (ES) m/e 329.0 (M+H)⁺; ¹H NMR (400 MHz, d₆-DMSO) □ 11.67 (s, 1H), 9.54 (s, 1H), 8.33 (d, J=8.2 Hz, 1H), 8.07 (d, J=1.8 Hz, 1H), 7.82 (s, 1H), 7.58 (d, J=8.7 Hz, 1H), 7.46 (d, J=8.2 Hz, 1H), 7.40 (dd, J=2.0, 8.7 Hz, 1H), 2.96 (s, 3H).

### Example 3

### N-[7-(trifluoromethyl)-9H-carbazol-3-yl]urea

To a stirred solution of 7-(trifluoromethyl)-9H-carbazol-3-amine from Example 1b above (100 mg, 0.40 mMol) in HOAc (5 mL) was added potassium cyanate (97 mg, 1.2 mMol) and water (100 uL). The reaction was stirred at RT for 18 h then evaporated to dryness under vacuum. Purification by flash chromatography on silica gel (5 to 10% MeOH in CH₂Cl₂), followed by trituration with (1:1) ether, pet. ether, filtration and drying under vacuum gave the title compound (39.1 mg, 33%) as an off-white solid: MS (ES) m/e 294.2 (M+H)⁺; ¹H NMR (400 MHz, d₆-DMSO) □ 11.45 (s, 1H), 8.53 (s, 1H), 8.27 (d, J=1.6 Hz, 1H), 8.23 (d, J=8.2 Hz, 1H), 7.77 (s, 1H), 7.47 (d, J=8.7 Hz, 1H), 7.42 (dd, 1H), 7.40 (d, 1H), 5.82 (s, 2H).

### Example 4

### N-[7-(Trifluoromethyl)-9H-carbazol-3-yl]thiourea

To a stirred solution of 7-(trifluoromethyl)-9H-carbazol-3-amine from Example 1b above (250 mg, 1.0 mMol) in acetone (5 mL) was added with stirring benzoyl isothiocyanate (135 uL, 1.0 mMol). The reaction was heated to 60° C. and stirred for 30 min. (LCMS showed complete conversion to the benzylthiourea). To the reaction was next added a solution of 5% NaOH in water (3 mL, 3.75 mMol), then reaction heated to 80° C. for an additional 30 minutes. The reaction was cooled in an ice bath, neutralized with 1 N HCl (3.75 mL), diluted with water, extracted with EtOAc, washed with brine, dried (MgSO₄), and concentrated under vacuum. Purification by flash chromatography on silica gel (30 to 50% EtOAc in CH₂Cl₂), followed by trituration with hexane, filtration and drying under vacuum gave the title compound (277 mg, 89%) as an off-white solid: MS (ES) m/e 310.2 (M+H)⁺; ¹H NMR (400 MHz, d₆-DMSO) □ 11.66 (s, 1H), 9.68 (s, 1H), 8.35 (d, J=8.2 Hz, 1H), 8.14 (s, 1H), 7.82 (s, 1H), 7.56 (d, J=8.6 Hz, 1H), 7.46 (dd, J=1.0, 8.2 Hz, 1H), 7.36 (dd, J=1.0, 8.2 Hz, 1H).

### Example 5

### N-[7-(trifluoromethyl)-9H-carbazol-3-yl]sulfamide

To a stirred solution of chlorosulfonyl isocyanate (0.5 mL, 5.7 mMol) in acetonitrile (10 mL) at 40° C. was added dropwise a solution of water (103 uL, 5.7 mMol) in acetonitrile (5 mL). After the addition, the reaction was allowed to warm to RT and stirred for 4 h. To the clear solution, with stirring at 0° C., was next added dropwise a solution of 7-(trifluoromethyl)-9H-carbazol-3-amine from Example 1b above (1.0 g, 4.0 mMol), pyridine (0.65 mL, 8.0 mMol) and dioxane (30 mL). The reaction was then allowed to warm to RT and stirred for 18 h. The reaction was concentrated under vacuum, taken up in EtOAc, washed with 1 N HCl, brine, dried (MgSO₄) and evaporated to dryness. Purification by flash chromatography on silica gel (30 to 50% EtOAc in CH₂Cl₂), followed by trituration with EtOAc, hexanes, filtration and drying under vacuum gave the title compound (426 mg, 32%) as a white solid: MS (ES) m/e 330.2 (M+H)⁺; ¹H NMR (400 MHz, d₆-DMSO) □ 11.55 (s, 1H), 9.23 (s, 1H), 8.25 (d, J=8.2 Hz, 1H), 8.02 (d, J=2.0 Hz, 1H), 7.80 (s, 1H), 7.52 (d, J=8.7 Hz, 1H), 7.45 (dd, J=1.2, 8.2 Hz, 1H), 7.35 (dd, J=2.1, 8.7 Hz, 1H), 6.98 (s, 2H).

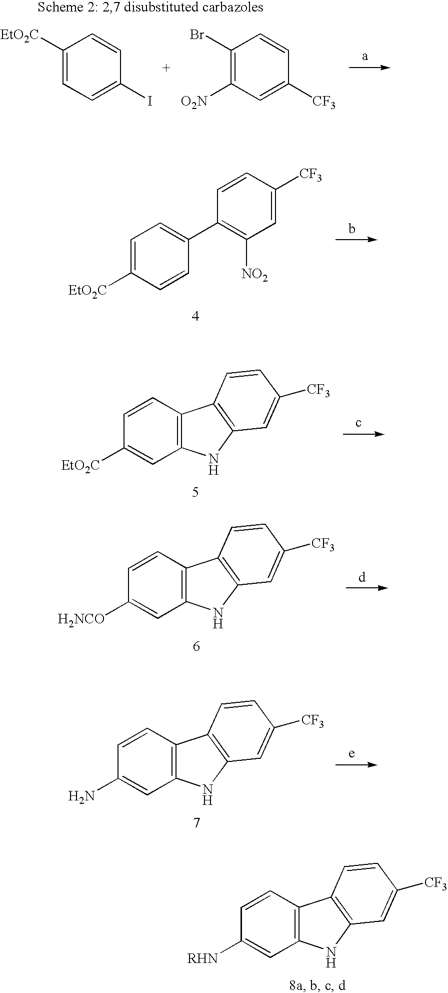

Conditions: a) Cu 180° C.; b) P(OEt)₃ reflux; c) NH₃, MeOH 120° C.; d) Br₂, 1 N NaOH, dioxane; e) For R=CH₃SO₂— (8a) CH₃SO₂Cl, pyridine, CH₂Cl₂; For R=H₂NCO—(8b) KOCN, HOAc; For R=H₂NCS— (8c) benzoyl isothiocyanate, acetone; 5% NaOH; For R=H₂NSO₂— (8d) H₂NSO₂Cl, pyridine, CH₃CN, dioxane

### Example 6

### 7-(trifluoromethyl)-9H-carbazol-2-ylamine

### 6a. Ethyl 2′-nitro-4′-(trifluoromethyl)-4-biphenylcarboxylate

A mixture of Ethyl 4-iodobenzoate (10.0 g, 36.2 mMol), 4-bromo-3-nitrobenzotrifluoride (5.0 g, 18.5 mmol) and Copper powder (5.0 g, 78.7 mmol) was heated to reflux (160-180° C.) and stirred for 4 h under Ar. The reaction was cooled to RT, diluted with EtOAc, filtered through a pad of Celite® and rinsed with EtOAc to remove the insolubles. The filtrate was concentrated under vacuum and purified by flash chromatography on silica gel (10% EtOAc in hexanes). Trituration with hexane, filtration and drying under vacuum gave the title compound (3.54 g, 56%) as a yellow solid: MS (ES) m/e 340.2 (M+H)⁺; ¹H NMR (400 MHz, CDCl₃) □ 8.22 (s, 1H), 8.16 (d, J=8.5 Hz, 2H), 7.94 (d, J=8.0 Hz, 1H), 7.63 (d, J=8.0 Hz, 1H), 7.42 (d, J=8.5 Hz, 2H), 4.44 (q, 2H), 1.44 (t, 3H).

### 6b. Ethyl 7-(trifluoromethyl)-9H-carbazole-2-carboxylate

A stirred solution of ethyl 2′-nitro-4′-(trifluoromethyl)-4-biphenylcarboxylate (5.0 g, 14.7 mMol) and triethyl phosphite (50 mL) was heated to reflux (170° C. oil bath) for 8 h. The now clear brown solution was cooled to RT and short path distilled under vacuum to remove most of the triethyl phosphite and triethyl phosphate by-product. The residue which remained was purified by flash chromatography on silica gel (10 to 50% EtOAc in hexanes), triturated with hexane, filtered and dried under vacuum to give the title compound (1.60 g, 35%) as a white solid: MS (ES) m/e 308.2 (M+H)⁺; ¹H NMR (400 MHz, d₆-DMSO) □ 8.44 (d, J=8.2 Hz, 1H), 8.37 (d, J=8.2 Hz, 1H), 8.22 (s, 1H), 7.93 (s, 1H), 7.85 (dd, J=1.4, 8.2 Hz, 1H), 7.53 (d, J=8.2 Hz, 1H), 4.38 (q, 2H), 1.38 (t, 3H).

### 6c. 7-(Trifluoromethyl)-9H-carbazole-2-carboxamide

To ethyl 7-(trifluoromethyl)-9H-carbazole-2-carboxylate (1.7 g, 5.5 mMol) in a pressure tube was added a solution of 7M ammonia in methanol (50 mL). The tube was sealed, heated to 120° C. and stirred for 3 days. After cooling the tube was opened and the reaction concentrated to dryness. Trituration with (1:1) ether, pet. ether, filtration and drying under vacuum gave the title compound (1.48 g, 96%) as a white solid: MS (ES) m/e 279.2 (M+H)⁺; ¹H NMR (400 MHz, d₆-DMSO) □ 12.01 (s, 1 H), 8.40 (d, J=8.2 Hz, 1H), 8.29 (d, J=8.2 Hz, 1H), 8.12 (s, 2H), 7.87 (s, 1H), 7.77 (dd, J=1.1, 8.3 Hz, 1H), 7.50 (d, J=8.2 Hz, 1H), 7.43 (s, 1H), 4.02 (q, 2H), 1.17 (t, 3H).

### 6d. 7-(trifluoromethyl)-9H-carbazole-2-ylamine

To a stirred solution of 7-(trifluoromethyl)-9H-carbazole-2-carboxamide (2.0 g, 7.2 mMol) suspended in dioxane (40 mL) was added dropwise a freshly prepared solution sodium hypobromite; prepared by adding bromine (0.4 mL, 7.8 mMol) dropwise to a 1 N NaOH solution (40 mL) at 0° C. and stirring until all the bromine was dissolved. The reaction cleared up during the addition. The reaction was stirred for 18 h at RT and concentrated under vacuum. After adjusting the pH to 7-8 with HOAc and diluting with water the resulting suspension was filtered, washed with water, then dried under vacuum to give the crude product. Purification by flash chromatography on silica gel (50 to 60% EtOAc in hexanes), followed by trituration with hexane, filtration and drying under vacuum gave the title compound (732 mg, 41%) as an off-white solid: MS (ES) m/e 251.2 (M+H)⁺; ¹H NMR (400 MHz, d₆-DMSO) □11.10 (s, 1H), 7.99 (d, J=8.1 Hz, 1H), 7.80 (d, J=8.4 Hz, 1H), 7.57 (s, 1H), 7.32 (d, J=8.1 Hz, 1H), 6.63 (d, J=1.7 Hz, 1H), 6.53 (dd, J=1.9, 8.4 Hz, 1H), 5.43 (s, 2H).

### Example 7

### N-[7-(trifluoromethyl)-9H-carbazol-2-yl]methanesulfonamide

According to the general procedure of compound 2 above, 2-amino-7-(trifluoromethyl)-9H-carbazole from Example 6d above (120 mg, 0.48 mMol) was converted to the title compound (145 mg, 98%) as a white solid: MS (ES) m/e 329.2 (M+H)⁺; ¹H NMR (400 MHz, d₆-DMSO) □ 11.63 (s, 1H), 9.94 (s, 1H), 8.26 (d, J=8.2 Hz, 1H), 8.17 (d, J=8.4 Hz, 1H), 7.77 (s, 1H), 7.47 (s, 1H), 7.46 (d, 1H), 7.11 (dd, J=1.8, 8.4 Hz, 1H), 3.03 (s, 3H).

### Example 8

### N-[7-(trifluoromethyl)-9H-carbazol-2-yl]urea

According to the general procedure of compound 3 above, 2-amino-7-(trifluoromethyl)-9H-carbazole from Example 6d above (150 mg, 0.60 mMol) was converted to the title compound (86 mg, 49%) as a white solid: MS (ES) m/e 294.2 (M+H)⁺; ¹H NMR (400 MHz, d₆-DMSO) □ 11.45 (s, 1H), 8.82 (s, 1H), 8.17 (d, J=8.2 Hz, 1H), 8.02 (d, J=8.5 Hz, 1H), 7.98 (d, J=1.6 Hz, 1H), 7.70 (s, 1H), 7.40 (dd, J=1.0, 8.2 Hz, 1H), 7.01 (dd, J=1.8, 8.5 Hz, 1H), 5.94 (s, 2H).

### Example 9

### N-[7-(trifluoromethyl)-9H-carbazol-2-yl]thiourea

According to the general procedure of compound 4 above, 2-amino-7-(trifluoromethyl)-9H-carbazole from Example 6d above (150 mg, 0.60 mMol) was converted to the title compound (108 mg, 58%) as a white solid: MS (ES) m/e 310.0 (M+H)⁺; ¹H NMR (400 MHz, d₆-DMSO) □ 11.64 (s, 1H), 9.91 (s, 1H), 8.28 (d, J=8.2 Hz, 1H), 8.15 (d, J=8.4 Hz, 1H), 7.83 (s, 1H), 7.78 (s, 1H), 7.60 (br s, 2H), 7.45 (dd, J=1.0, 8.2 Hz, 1H), 7.13 (dd, J=1.8, 8.4 Hz, 1H).

### Example 10

### N-[7-(trifluoromethyl)-9H-carbazol-2-yl]sulfamide

According to the general procedure of compound 5 above, 2-amino-7-(trifluoromethyl)-9H-carbazole from Example 6d above (0.85 g, 3.4 mMol) was converted to the title compound (471 mg, 42%) as a white solid: MS (ES) m/e 330.2 (M+H)⁺; ¹H NMR (400 MHz, d₆-DMSO) □ 11.54 (s, 1H), 9.70 (s, 1H), 8.21 (d, J=8.1 Hz, 1H), 8.09 (d, J=8.5 Hz, 1H), 7.72 (s, 1H), 7.43 (dd, J=1.1, 8.3 Hz, 1H), 7.41 (d, J=1.7 Hz, 1H), 7.17 (s, 2H), 7.06 (dd, J=1.9, 8.5 Hz, 1H).

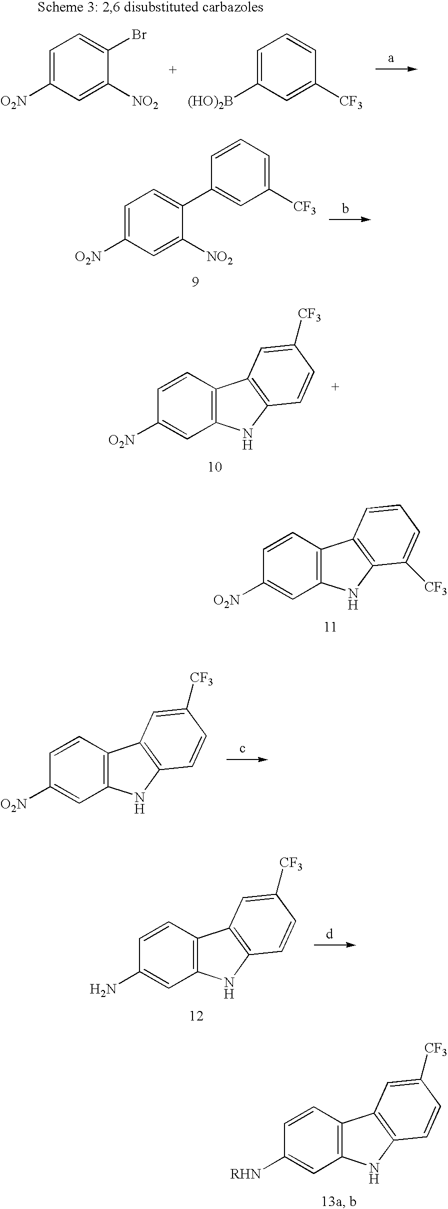

Conditions: a) Pd(PPh₃)₄, K₂CO₃, H₂O, dioxane 80° C. 18 h; b) P(OEt)₃, cumene reflux 2 h; c) Zn, HOAc; d) For R=H₂NCO— (13a) KOCN, HOAc; For R=H₂NSO₂— (13b) H₂NSO₂Cl, pyridine, CH₃CN, dioxane.

### Example 11

### 6-(trifluoromethyl)-9H-carbazol-2-ylamine

### 11a. 2,4-Dinitro-3′-(trifluoromethyl)biphenyl

To a stirred solution of 1-bromo-2,4-dinitrobenzene (10 g, 40 mMol) and 3-trifluoromethylphenylboronic acid (8.25 g, 43 mMol) in dioxane (100 mL) was added a solution of 2 M K₂CO₃ in water (44 mL) followed by Pd(PPh₃)₄ (1.0 g, 0.86 mMol). The reaction was purged with N₂, heated to 80° C. in an oil bath, and stirred for 18 h. After cooling to RT, the reaction was concentrated under vacuum, took up in EtOAc, filtered to remove insolubles, washed with water, brine, dried (MgSO₄) and evaporated. Purification by flash chromatography on silica gel (10 to 20% EtOAc in hexanes), followed by trituration with hexane, filtration and drying under vacuum gave the title compound (12.14 g, 96%) as a yellow solid: MS (ES) m/e 313.2 (M+H)⁺; ¹H NMR (400 MHz, d₆-DMSO) q 8.88 (d, J=2.3 Hz, 1H), 8.60 (dd, J=2.3, 8.5 Hz, 1H), 7.95 (d, J=8.5 Hz, 1H), 7.89 (m, 1H), 7.87 (s, 1H), 7.74-7.79 (m, 2H).

### 11b. 2-Nitro-6-(trifluoromethyl)-9H-carbazole

To a stirred solution of 2,4-dinitro-3′-(trifluoromethyl)biphenyl (7.0 g, 22.4 mMol) in cumene (35 mL) was added triethylphosphite (12 mL, 70 mMol). The reaction was heated to reflux (170° C. oil bath) and stirred for 2 h. The reaction was cooled to RT, concentrated under vacuum and purified by flash chromatography on silica gel (10 to 20% EtOAc in hexanes) to give the title compound (0.76 g, 12%) as a yellow solid: MS (ES) m/e 281.0 (M+H)⁺; ¹H NMR (400 MHz, d₆-DMSO) □ 12.24 (s, 1H), 8.78 (s, 1H), 8.55 (d, J=8.7 Hz, 1H), 8.44 (d, J=2.0 Hz, 1H), 8.10 (dd, J=2.1, 8.7 Hz, 1H), 7.83 (dd, J=1.6, 8.7 Hz, 1H), 7.80 (d, J=8.7 Hz, 1H).

A second faster running regioisomer, 2-nitro-8-(trifluoromethyl)-9H-carbazole (11), was also isolated (1.05 g, 16%) as a yellow solid: MS (ES) m/e 281.0 (M+H)⁺; ¹H NMR (400 MHz, d₆-DMSO) □ 12.15 (s, 1H), 8.62 (d, J=7.9 Hz, 1H), 8.47 (d, J=8.7 Hz, 1H), 8.44 (d, J=2.0 Hz, 1H), 8.12 (dd, J=2.1, 8.7 Hz, 1H), 7.90 (d, J=7.6 Hz, 1H), 7.45 (app. t, J=7.6 Hz, 1H).

### 11c. 6-(trifluoromethyl)-9H-carbazol-2-ylamine

To a stirred solution of 2-nitro-6-(trifluoromethyl)-9H-carbazole (0.83 g, 3.0 mMol) in HOAc (30 mL) was added portionwise zinc powder (1.36 g, 21 mMol). The reaction was stirred at RT for 18 h, filtered through a pad of Celite, rinsed with HOAc, and concentrated under vacuum. The residue was dissolved in EtOAc, washed with 1 N Na₂CO₃, brine, dried (Na₂SO₄) and evaporated to dryness. Purification by flash chromatography on silica gel (50 to 60% EtOAc in hexanes), followed by trituration with hexane, filtration and drying under vacuum gave the title compound (381 mg, 50%) as an off-white solid: MS (ES) m/e 251.0 (M+H)⁺; ¹H NMR (400 MHz, d₆-DMSO) □11.90 (s, 1H), 8.20 (s, 1H), 7.85 (d, J=8.4 Hz, 1H), 7.46 (dd, J=1.6, 8.6 Hz, 1H), 7.43 (d, J=8.5 Hz, 1H), 6.63 (d, J=1.7 Hz, 1H), 6.53 (dd, J=1.8, 8.4 Hz, 1H), 5.36 (s, 2H).

### Example 12

### N-[6-(trifluoromethyl)-9H-carbazol-2-yl]urea

According to the general procedure of compound 2 above, 2-amino-6-(trifluoromethyl)-9H-carbazole from Example 11c above (150 mg, 0.6 mMol) was converted to the title compound (132 mg, 75%) as a white solid: MS (ES) m/e 294.2 (M+H)⁺; ¹H NMR (400 MHz, d₆-DMSO) □ 11.52 (s, 1H), 8.78 (s, 1H), 8.40 (s, 1H), 8.07 (d, J=8.4 Hz, 1H), 7.97 (d, J=1.6 Hz, 1H), 7.58 (dd, J=1.5, 8.6 Hz, 1H), 7.55 (d, J=8.5 Hz, 1H), 7.00 (dd, J=1.8, 8.5 Hz, 1H), 5.92 (s, 2H).

### Example 13

### N-[6-(trifluoromethyl)-9H-carbazol-2-yl]sulfamide

According to the general procedure of compound (3d), 2-amino-6-(trifluoromethyl)-9H-carbazole from Example 11c above (200 mg, 0.8 mMol) was converted to the title compound (73 mg, 27%) as a white solid: MS (ES) m/e 330.2 (M+H)⁺; ¹H NMR (400 MHz, d₆-DMSO) □ 11.62 (s, 1H), 9.66 (s, 1H), 8.44 (s, 1H), 8.15 (d, J=8.5 Hz, 1H), 7.61 (dd, J=1.6, 8.5 Hz, 1H), 7.58 (d, J=8.5 Hz, 1H), 7.39 (d, J=1.6 Hz, 1H), 7.15 (s, 2H), 7.06 (dd, J=1.9, 8.5 Hz, 1H).

Examples 14 and 15 are prepared as shown generally in scheme 4.

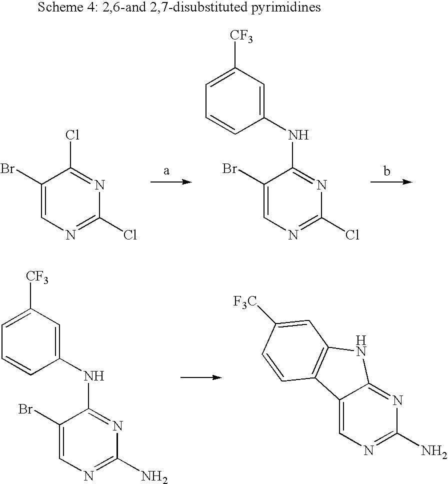

Conditions: a) 4-CF₃-aniline (for Example 14) or 3-CF₃-aniline (for Example 15), NaHCO₃, EtOH, 70° C.; b) NH₄OH, EtOH, sealed tube, 80° C.; c) Pd(OAc)₂, PPh₃, Ag₂CO₃, DMF, 150° C.

### Example 14

### 7-(trifluoromethyl)-1H-pyrimido[4,5-b]indol-2-ylamine

### 14a. 5-Bromo-2-chloro-N-[3-(trifluoromethyl)phenyl]-4-pyrimidinamine

To a stirred solution of 5-bromo-2,4-dichloropyrimidine (5.47 g, 21.9 mmol) in dry EtOH (40 mL) was added 3-(trifluoromethyl)aniline (3.5 g, 21.9 mmol) followed by sodium bicarbonate (2.0 g, 24.1 mmol). The resultant mixture was heated to 70° C. for 16 h. The mixture was filtered, concentrated under reduced pressure and the residue was purified by silica gel chromatography (9:1 hexanes/EtOAc) to give 6.8 g (72%) 5-bromo-2-chloro-N-[3-(trifluoromethyl)phenyl]-4-pyrimidinamine contaminated with ˜20% of 5-bromo-N,N′-bis[3-(trifluoromethyl)phenyl]-2,4-pyrimidinediamine as a white tacky solid. The mixture was carried on to the next step without further purification. 14b. To a stirred solution of 5-bromo-2-chloro-N-[3-(trifluoromethyl)phenyl]-4-pyrimidinamine contaminated with ˜20% of 5-bromo-N,N′-bis[3-(trifluoromethyl)phenyl]-2,4-pyrimidinediamine (6.8 g) in EtOH (150 mL) was added concentrated NH₄OH (100 mL). The reaction vessel was sealed and heated to 80° C. After 72 h, the reaction mixture was concentrated under reduced pressure and the residue was purified by silica gel chromatography (5:1 to 2:1 hexanes/EtOAc) to give 3.8 g (59%) of 5-bromo-N⁴-[3-(trifluoromethyl)phenyl]-2,4-pyrimidinediamine as a white solid. ESMS [M+H]⁺: 333.2.

14c. To a stirred solution of 5-bromo-N⁴-[3-(trifluoromethyl)phenyl]-2,4-pyrimidinediamine (300 mg, 0.902 mmol) in dry DMF (9 mL) was added palladium(II) acetate (203 mg, 0.902 mmol), triphenylphosphine (472 mg, 1.80 mmol) and silver carbonate (496 mg, 1.80 mmol) successively under an atmosphere of argon. The reaction mixture was heated to 150° C. for 18 h, allowed to cool to room temperature and diluted with H₂O (20 mL) and EtOAc (25 mL). The aqueous layer was extracted with two additional 25 mL portions of EtOAc and the combined extracts were dried (Na₂SO₄), filtered and concentrated under reduced pressure. The residue was purified by silica gel chromatography (1:3 hexanes/EtOAc) followed by purification by reverse phase chromatography (MeCN/H₂O/TFA) to give 12.2 mg (5%) of the title compound as a white solid. ESMS [M+H]⁺: 253.0. ¹H NMR (400 MHz, dmso-d₆): □ 12.63 (s, 1H), 9.18 (s, 1H), 8.23 (d, J=8.0 Hz, 1H), 7.88 (broadened s, 2H), 7.67 (s, 1H), 7.63 (d, J=4.0 Hz, 1H).

### Example 15

### 6-(Trifluoromethyl)-1H-pyrimido[4,5-b]indol-2-ylamine

Substituting 3-(trifluoromethyl)aniline in place of 4(trifluoromethyl)aniline and utilizing the general procedure described for Example 14, the title compound was prepared as a white solid. ESMS [M+H]⁺: 253.2. ¹H NMR (400 MHz, dmso-d₆): □12.44 (s, 1H), 8.96 (s, 1H), 8.30 (s, 1H), 7.65 (broadened s, 2H), 7.56 (d, J=1.6 Hz, 1H), 7.40 (d, J=8.0 Hz, 1H).

### Example 16

### 16a. 6-(Trifluoromethyl)-9H-carbazol-3-ylamine

### 16a. 6-(Trifluoromethyl)-2,3,4,4a,9,9a-hexahydro-1H-carbazole

To (4-trifluoromethyl-phenyl)-hydrazine (4.71 g, 26.74 mmol) in ethanol (20 mL) was added concentrated hydrochloric acid (10 drops) followed by a solution of cyclohexanone (2.62 g, 26.74 mmol) in ethanol (4 mL). The reaction was stirred at ro temperature for 45 minutes and concentrated in vacuo. The resulting solid was dissolved in a mixture of acetic acid (21 mL) and concentrated sulfuric acid (3.5 mL). The reaction was heated at 85° C. for 30 minutes, cooled to room temperature, and poured into ice water. The resulting suspension was filtered and dried to give the title compound as a tan solid (6.01 g, 94%). MS (ES+) m/e 240 [M+H]⁺. 16b. 3-Trifluoromethyl-9H-carbazole

To 6-trifluoromethyl-2,3,4,9-tetrahydro-1H-carbazole (7.12 g, 29.76 mmol) in xylenes (150 mL) was added 2,3-dichloro-5,6-dicyano-1,4-benzoquinone (13.51 g, 59.52 mmol). The reaction was stirred and heated at 140° C. for 1.5 hours, cooled to room temperature, diluted with ether, and filtered. The filtered solid was purified by flash chromatography on silica gel (3:1 hexanes:EtOAc) to give the title compound as a tan solid (2.69 g, 38%). MS (ES+) m/e 236 [M+H]⁺.

### 16c. 3-Nitro-6-trifluoromethyl-9H-carbazole

To 3-trifluoromethyl-9H-carbazole (1.18 g, 5.03 mmol) in acetic acid (35 mL) was added fuming nitric acid (0.33 g, 5.284 mmol). The reaction was stirred at 60° C. for 4 hr and concentrated in vacuo. The residue was suspended between EtOAc/saturated NaCl, washed with saturated NaCl, dried (Na₂SO₄), and evaporated to dryness. The residue was purified by flash chromatography on silica gel (1:1 hexanes:EtOAc to remove the 1-nitro regioisomer followed by elution with 25% MeOH:CH₂Cl₂ to give the title compound as a yellow solid (0.765 g, 55%). MS (ES+) m/e 281 [M+H]⁺.

### 16d. 6-(Trifluoromethyl)-9H-carbazol-3-amine

To 3-nitro-6-trifluoromethyl-9H-carbazole (0.762 g, 2.72 mmol) in ethanol (40 mL) was added 10% Pd-carbon (80 mg). The mixture was placed under a balloon filled with hydrogen and stirred overnight at room temperature. The reaction was filtered through Celite®, concentrated, and purified by flash chromatography on silica gel (2:1 hexanes:EtOAc) to give the title compound as a rust colored solid (0.348 g, 51%). MS (ES+) m/e 251 [M+H]⁺.

Inhibition of Cellular Viability in Tumor Cell Lines Treated with KSP Inhibitors

### Materials and Solutions:

Cells: SKOV3, Ovarian Cancer (human).

Media: Phenol Red Free RPMI+5% Fetal Bovine Serum+2 mM L-glutamine.

Colorimetric Agent for Determining Cell Viability: Promega MTS tetrazolium compound.

Control Compound for max cell kill: Topotecan, 1 μM.

Procedure: Day 1—Cell Plating:

Adherent SKOV3 cells are washed with 10 mLs of PBS followed by the addition of 2 mLs of 0.25% trypsin and incubation for 5 minutes at 37° C. The cells are rinsed from the flask using 8 mL of media (phenol red-free RPMI+5% FBS) and transferred to fresh flask. Cell concentration is determined using a Coulter counter and the appropriate volume of cells to achieve 1000 cells/100 μL is calculated. 100 μL of media cell suspension (adjusted to 1000 cells/100 μL) is added to all wells of 96-well plates, followed by incubation for 18 to 24 hours at 37° C., 100% humidity, and 5% CO₂, allowing the cells to adhere to the plates.

Procedure: Day 2—Compound Addition:

To one column of the wells of an autoclaved assay block are added an initial 2.5 μL of test compound(s) at 400× the highest desired concentration. 1.25 μL of 400× (400 μM)

Topotecan is added to other wells (ODs from these wells are used to subtract out for background absorbance of dead cells and vehicle). 500 μL of media without DMSO are added to the wells containing test compound, and 250 μL to the Topotecan wells. 250 μL of media+0.5% DMSO is added to all remaining wells, into which the test compound(s) are serially diluted. By row, compound-containing media is replica plated (in duplicate) from the assay block to the corresponding cell plates. The cell plates are incubated for 72 hours at 37° C., 100% humidity, and 5% CO₂.

Procedure: Day 4—MTS Addition and OD Reading:

The plates are removed from the incubator and 40 μl MTS/PMS is added to each well. Plates are then incubated for 120 minutes at 37° C., 100% humidity, 5% CO₂, followed by reading the ODs at 490 nm after a 5 second shaking cycle in a ninety-six well spectrophotometer.

### Data Analysis

The normalized % of control (absorbance−background) is calculated and an XLfit is used to generate a dose-response curve from which the concentration of compound required to inhibit viability by 50% is determined. The compounds of the present invention show activity when tested by this method.

### Example 17

### Monopolar Spindle Formation following Application of a KSP Inhibitor

Human tumor cells Skov-3 (ovarian) were plated in 96-well plates at densities of 4,000 cells per well, allowed to adhere for 24 hours, and treated with various concentrations of the test compounds for 24 hours. Cells were fixed in 4% formaldehyde and stained with antitubulin antibodies (subsequently recognized using fluorescently-labeled secondary antibody) and Hoechst dye (which stains DNA).

Visual inspection revealed that the compounds caused cell cycle arrest in the prometaphase stage of mitosis. DNA was condensed and spindle formation had initiated, but arrested cells uniformly displayed monopolar spindles, indicating that there was an inhibition of spindle pole body separation. Microinjection of anti-KSP antibodies also causes mitotic arrest with arrested cells displaying monopolar spindles.

### Example 18

### Inhibition of Cellular Proliferation in Tumor Cell Lines Treated with KSP Inhibitors

Cells were plated in 96-well plates at densities from 1000-2500 cells/well of a 96-well plate and allowed to adhere/grow for 24 hours. They were then treated with various concentrations of drug for 48 hours. The time at which compounds are added is considered T₀. A tetrazolium-based assay using the reagent 3-(4,5-dimethylthiazol-2-yl)-5-(3-carboxymethoxyphenyl)-2-(4-sulfophenyl)-2H-tetrazolium (MTS) (U.S. Pat. No. 5,185,450) (see Promega product catalog #G3580, CellTiter 96® AQᵤₑₒᵤₛ One Solution Cell Proliferation Assay) was used to determine the number of viable cells at T₀ and the number of cells remaining after 48 hours compound exposure. The number of cells remaining after 48 hours was compared to the number of viable cells at the time of drug addition, allowing for calculation of growth inhibition.

The growth over 48 hours of cells in control wells that had been treated with vehicle only (0.25% DMSO) is considered 100% growth and the growth of cells in wells with compounds is compared to this. KSP inhibitors inhibited cell proliferation in human ovarian tumor cell lines (SKOV-3).

An IC₅₀ was calculated by plotting the concentration of compound in μM vs the percentage of cell growth of cell growth in treated wells. The IC₅₀ calculated for the compounds is the estimated concentration at which growth is inhibited by 50% compared to control, i.e., the concentration at which:

100×[(Treated₄₈−T₀)/(Control₄₈−T₀)]=50.

All concentrations of compounds are tested in duplicate and controls are averaged over 12 wells. A very similar 96-well plate layout and IC₅₀ calculation scheme is used by the National Cancer Institute (see Monks, et al., J. Natl. Cancer Inst. 83:757-766 (1991)). However, the method by which the National Cancer Institute quantitates cell number does not use MTS, but instead employs alternative methods.

### Example 19

### Calculation of IC₅₀

Measurement of a composition's IC₅₀ for KSP activity uses an ATPase assay. The following solutions are used: Solution 1 consists of 3 mM phosphoenolpyruvate potassium salt (Sigma P-7127), 2 mM ATP (Sigma A-3377), 1 mM DTT (Sigma D-9779), 5 μM paclitaxel (Sigma T-7402), 10 ppm antifoam 289 (Sigma A-8436), 25 mM Pipes/KOH pH 6.8 (Sigma P6757), 2 mM MgC12 (VWR JT400301), and 1 mM EGTA (Sigma E3889). Solution 2 consists of 1 mM NADH (Sigma N8129), 0.2 mg/ml BSA (Sigma A7906), pyruvate kinase 7 U/ml, L-lactate dehydrogenase 10 U/ml (Sigma P0294), 100 nM KSP motor domain, 50 μg/ml microtubules, 1 mM DTT (Sigma D9779), 5 μM paclitaxel (Sigma T-7402), 10 ppm antifoam 289 (Sigma A-8436), 25 mM Pipes/KOH pH 6.8 (Sigma P6757), 2 mM MgC12 (VWR JT4003-01), and 1 mM EGTA (Sigma E3889). Serial dilutions (8-12 two-fold dilutions) of the composition are made in a 96-well microtiter plate (Corning Costar 3695) using Solution 1. Following serial dilution each well has 50 μl of Solution 1. The reaction is started by adding 50 μl of solution 2 to each well. This may be done with a multichannel pipettor either manually or with automated liquid handling devices. The microtiter plate is then transferred to a microplate absorbance reader and multiple absorbance readings at 340 nm are taken for each well in a kinetic mode. The observed rate of change, which is proportional to the ATPase rate, is then plotted as a function of the compound concentration. For a standard IC₅₀ determination the data acquired is fit by the following four parameter equation using a nonlinear fitting program (e.g., Grafit 4):

$$y = \frac{Range}{1 + {( \frac{x}{{IC}_{50}} )}^{s}} + Background$$

where y is the observed rate and x the compound concentration.
Other compounds of this class were found to inhibit cell proliferation, although IC₅₀ values varied. IC₅₀ values for the compounds tested ranged from less than 200 nM to greater than the highest concentration tested. By this we mean that although most of the compounds that inhibited KSP activity biochemically did inhibit cell proliferation, for some, at the highest concentration tested (generally about 20 μM), cell growth was inhibited less than 50%. Many of the compounds have IC₅₀ values less than 10 μM, and several have IC₅₀ values less than 1 μM. Anti-proliferative compounds that have been successfully applied in the clinic to treatment of cancer (cancer chemotherapeutics) have IC₅₀'s that vary greatly. For example, in A549 cells, paclitaxel IC₅₀ is 4 nM, doxorubicin is 63 nM, 5-fluorouracil is 1 μM, and hydroxyurea is 500 μM (data provided by National Cancer Institute, Developmental Therapeutic Program, http://dtp.nci.nih.gov/). Therefore, compounds that inhibit cellular proliferation at virtually any concentration may be useful. However, preferably, compounds will have IC₅₀ values of less than 1 mM. More preferably, compounds will have IC₅₀ values of less than 20 μM. Even more preferably, compounds will have IC₅₀ values of less than 10 μM. Further reduction in IC₅₀ values may also be desirable, including compounds with IC₅₀ values of less than 1 μM. Some of the compounds of the invention inhibit cell proliferation with IC₅₀ values below 200 nM.

### Biological Activity of Compounds of Examples 1-16

Compound Examples 1-16 were found to inhibit KSP with an IC₅₀<10 uM. The compounds were also shown to inhibit the proliferation of various tumor cell lines including SKOV3 and Colo205 and typically had IC₅₀<50 uM. The compounds with formulae: N-[6-(trifluoromethyl). 9H-carbazol-1-yl]methanesulfonamide, 6-(trifluoromethyl)-9H-carbazol-1-ylamine, 6-(trifluoromethyl-9H-carbazol-3-yl)-methanesulfonamide, (6-Trifluoromethyl-9H-carbazol-3-yl)-urea were not active in the ranges listed above.

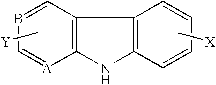

| Compound | Y | X | A | B |
|---|---|---|---|---|
| 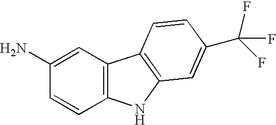 | 3-NH₂ | 7-CF₃ | CH | CH |
| Example 1: 7-(trifluoromethyl)-9H-carbazol-3- | | | | |
| ylamine | | | | |
| 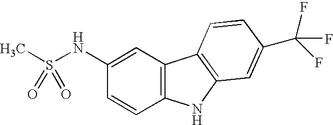 | 2-NHSO₂Me | 7-CF₃ | CH | CH |
| Example 2: N-[7-(trifluoromethyl)-9H-carbazol-3- | | | | |
| yl]methanesulfonamide | | | | |
| 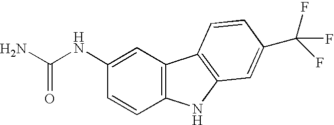 | 3-NHCONH₂ | 7-CF₃ | CH | CH |
| Example 3: N-[7-(trifluoromethyl)-9H-carbazol-3 | | | | |
| yl]urea | | | | |
| 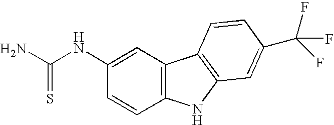 | 3-NHCSNH₂ | 7-CF₃ | CH | CH |
| Example 4: N-[7-(trifluoromethyl)-9H-carbazol-3- | | | | |
| yl]thiourea | | | | |
| 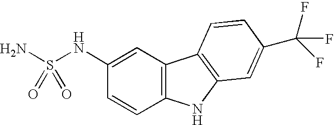 | 3-NHSO₂NH₂ | 7-CF₃ | CH | CH |
| Example 5: N-[7-(trifluoromethyl)-9H-carbazol-3- | | | | |
| yl]sulfamide | | | | |
| 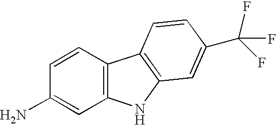 | 2-NH₂ | 7-CF₃ | CH | CH |
| Example 6: 7-(trifluoromethyl)-9H-carbazol-2- | | | | |
| ylamine | | | | |
| 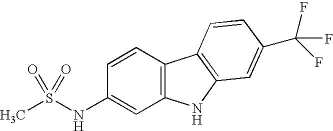 | 2-NHSO₂Me | 6-CF₃ | CH | CH |
| Example 7: N-[7-(trifluoromethyl)-9H-carbazol-2- | | | | |
| yl]methanesulfonamide | | | | |
| 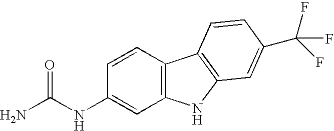 | 2-NHCONH₂ | 7-CF₃ | CH | CH |
| Example 8: N-[7-(trifluoromethyl)-9H-carbazol-2- | | | | |
| yl]urea | | | | |
| 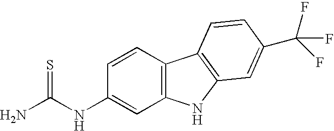 | 2-NHCSNH₂ | 7-CF₃ | CH | CH |
| Example 9: N-[7-(trifluoromethyl)-9H-carbazol-2- | | | | |
| yl]thiourea | | | | |
| 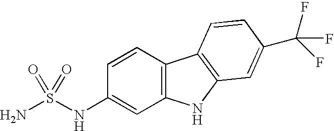 | 2-NHSO₂NH₂ | 7-CF₃ | CH | CH |
| Example 10: N-[7-(trifluoromethyl)-9H-carbazol- | | | | |
| 2-yl]sulfamide | | | | |
| 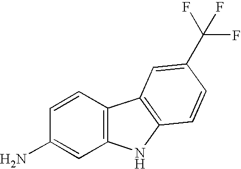 | 2-NH₂ | 6-CF₃ | CH | CH |
| Example 11: 6-(trifluoromethyl)-9H-carbazol-2- | | | | |
| ylamine | | | | |
| 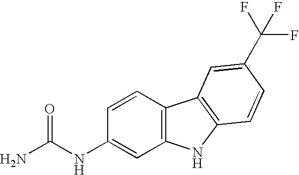 | 2-NHCONH₂ | 6-CF₃ | CH | CH |
| Example 12: N-[6-(trifluoromethyl)-9H-carbazol- | | | | |
| 2-yl]urea | | | | |
| 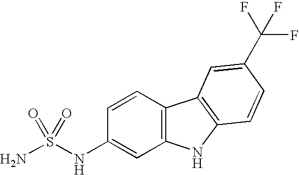 | 2-NHSO₂NH₂ | 6-CF₃ | CH | CH |
| Example 13: N-[6-(trifluoromethyl)-9H-carbazol- | | | | |
| 2-yl]sulfamide | | | | |
| 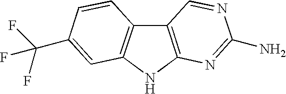 | 2-NH₂ | 7-CF₃ | N | N |
| Example 14: 7-(trifluoromethyl)-1H-pyrimido[4,5- | | | | |
| b]indol-2-ylamine | | | | |
| 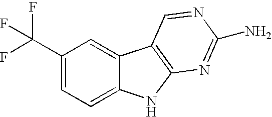 | 2-NH₂ | 6-CF₃ | N | N |
| Example 15: 6-(trifluoromethyl)-1H-pyrimido[4,5- | | | | |
| b]indol-2-ylamine | | | | |
| 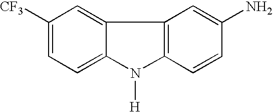 | 3-NH₂ | 6-CF₃ | CH | CH |
| Example 16: 6-(trifluoromethyl)-9H-carbazol-3 | | | | |
| amine | | | | |

N-[6-(trifluoromethyl)-9H-carbazol-1-yl]methanesulfonamide, 6-(trifluoromethyl)-9H-carbazol-1-ylamine, 6-(trifluoromethyl-9H-carbazol-3-yl)-methanesulfonamide, (6-Trifluoromethyl-9H-carbazol-3-yl)-urea

1. A compound of formula I: 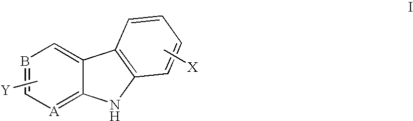
wherein:
X is selected from the group consisting of CF₃ and S(O)ₙR₁;
Y is selected from the group consisting of NR₁R₂, NR₁COR₂, NR₁CONR₂R₃, NR₁CSNR₂R₃, NR₁S(O)ₙNR₂R₃ and NR₁S(O)ₙR₂;
n is 1 or 2;
R₁, R₂ and R₃ are at each occurrence independently selected from a group consisting of hydrogen, optionally substituted alkyl, optionally substituted aryl, optionally substituted aralkyl, optionally substituted heteroaryl and optionally substituted heteroarylalkyl;
A and B are independently carbon or nitrogen; or
a pharmaceutically acceptable salt or solvate thereof.

2. A compound of claim 1 wherein X is CF₃.

3. A compound of claim 1 wherein X is S(O)ₙR₁.

4. A compound of claim 1 wherein A and B are carbon.

5. A compound of claim 1 wherein A and B are nitrogen.

6. A compound of claim 1 wherein Y is NR₁R₂.

7. A compound of claim 6 wherein R₁ and R₂ at each occurrence are independently hydrogen or optionally substituted alkyl.

8. A compound of claim 7 wherein R₁ and R₂ are hydrogen.

9. A compound of claim 1 wherein Y is NR₁COR₂.

10. A compound of claim 9 wherein R₁ and R₂ at each occurrence are independently optionally substituted alkyl.

11. A compound of claim 1 wherein Y is NR₁CONR₂R₃.

12. A compound of claim 11 wherein R₁, R₂ and R₃ at each occurrence are independently hydrogen or optionally substituted alkyl.

13. A compound of claim 1 wherein Y is NR₁CSNR₂R₃.

14. A compound of claim 13 wherein R₁, R₂ and R₃ at each occurrence are independently hydrogen or optionally substituted alkyl.

15. A compound of claim 1 wherein Y is NR₁Sₙ(O)ₙNR₂R₃.

16. A compound of claim 15 wherein R₁, R₂ and R₃ at each occurrence are independently hydrogen or optionally substituted alkyl.

17. A compound of claim 1 wherein Y is NR₁S(O)ₙR₂.

18. A compound of claim 17 wherein R₁ and R₂ at each occurrence are independently hydrogen or optionally substituted alkyl.

19. A compound of claim 18 wherein R₁ and R₂ at each occurrence are independently hydrogen or methyl.

20. A compound of claim 1 which is:
a) 7-(trifluoromethyl)-1H-pyrimido[4,5-b]indol-2-ylamine;
b) 6-(trifluoromethyl)-1H-pyrimido[4,5-b]indol-2-ylamine;
c) 6-(trifluoromethyl)-9H-carbazol-2-ylamine;
d) 6-(trifluoromethyl)-9H-carbazol-3-ylamine;
e) N-[7-(trifluoromethyl)-9H-carbazol-2-yl]methanesulfonamide;
f) N-[6-(trifluoromethyl)-9H-carbazol-2-yl]urea;
g) 7-(trifluoromethyl)-9H-carbazol-3-ylamine;
h) N-[7-(trifluoromethyl)-9H-carbazol-3-yl]urea;
i) N-[6-(trifluoromethyl)-9H-carbazol-2-yl]sulfamide;
j) N-[7-(trifluoromethyl)-9H-carbazol-3-yl]methanesulfonamide;
k) N-[7-(trifluoromethyl)-9H-carbazol-2-yl]urea;
l) N-[7-(trifluoromethyl)-9H-carbazol-3-yl]thiourea;
m) N-[7-(trifluoromethyl)-9H-carbazol-2-yl]thiourea;
n) 7-(trifluoromethyl)-9H-carbazol-2-ylamine;
o) N-[7-(trifluoromethyl)-9H-carbazol-2-yl]sulfamide; or
p) N-[7-(trifluoromethyl)-9H-carbazol-3-yl]sulfamide,
or a pharmaceutically acceptable salt or solvate thereof.

21. A composition comprising a pharmaceutically acceptable excipient and the compound, or a pharmaceutically acceptable salt or solvate thereof, of claim 1.

22. A composition of claim 21, wherein said composition further comprises a taxane.

23. A composition of claim 21, wherein said composition further comprises a vinca alkaloid.

24. A composition of claim 21, wherein said composition further comprises a topoisomerase I inhibitor.

25. A method of modulating KSP kinesin activity which comprises contacting said kinesin with an effective amount of the compound, or a pharmaceutically acceptable salt or solvate thereof, according to claim 1.

26. A method of inhibiting KSP which comprises contacting said kinesin with an effective amount of the compound, or a pharmaceutically acceptable salt or solvate thereof, according to claim 1.

27. A method for the treatment of a disease of proliferating cells comprising administering to a subject in need thereof the compound or composition, or a pharmaceutically acceptable salt or solvate thereof, according to claim 1.

28. A method according to claim 27 wherein said disease is selected from the group consisting of cancer, hyperplasias, restenosis, cardiac hypertrophy, immune disorders, fungal disorders and inflammation.
